(19) United States
(12) Patent Application Publication
(10) Pub. No.: US 2001/0019834 A1
(43) Pub. Date: Sep. 6, 2001

(54) RECOMBINANT MICROORGANISMS EXPRESSING ANTIGENIC PROTEINS OF HELICOBACTER PYLORI

(76) Inventors: BYUNG-O KIM, SUNGNAM (KR); SUNG-SEUP SHIN, KIMPO (KR); YOUNG-HYO YU, KUNPO (KR); MYUNG-HWAN PARK, SEOUL (KR); DEOK-JOON CHOI, SUNGNAM (KR); HYUNG-JIN JUNG, SEOUL (KR)

Correspondence Address: DARBY & DARBY, 805 THIRD AVENUE, NEW YORK, NY, 10022

(21) Appl. No.: 09/402,100

(22) PCT Filed: Mar. 31, 1998
(86) PCT No.: PCT/KR98/00073

§ 371 Date: None

(30) Foreign Application Priority Data
Mar. 31, 1997 (KR) 97-11950
Mar. 31, 1997 (KR) 97-11951

(51) Int. Cl. C12N 15/09; A61K 39/02

(52) U.S. Cl. 435/69.2; 424/234.1

(57) ABSTRACT

The present invention relates to chimeric proteins consisting of antigenic proteins of Helicobacter pylori and A2 and B subunits of Vibrio cholerae toxin, more specifically, to recombinant DNAs coding for antigenic proteins Helicobacter pylori and A2 and B subunits of Vibrio cholerae toxin, recombinant expression vectors containing the genes, a process for preparing the chimeric proteins employing the recombinant microorganisms transformed with the said expression vectors, and preventive and therapeutic vaccines comprising the chimeric proteins for Helicobacter pylori-associated diseases. The recombinant DNAs which are designed for convenient expression and gene manipulation, can express chimeric proteins having excellent immunogenicity to H. pylori, which are stable in stomach, and penetrate mucous membrane of intestines easily, finally to stimulate production of sIgA. Accordingly, the chimeric proteins expressed from the recombinant DNAs may be used as an active ingredient of the diagnostic kit for H. pylori infection and preventive or therapeutic vaccine for H. pylori-associated diseases, and may be used in the production of anti-H. pylori antibody.

### BACKGROUND OF THE INVENTION

1. Field of the Invention

The present invention relates to chimeric proteins consisting of antigenic proteins of Helicobacter pylori and A2 and B subunits of Vibrio cholerae toxin, more specifically, to recombinant DNAs coding for antigenic proteins of Helicobacter pylori and A2 and B subunits of Vibrio cholerae toxin, recombinant expression vectors containing the genes, a process for preparing the chimeric proteins employing the recombinant microorganisms transformed with the said expression vectors, and preventive and therapeutic vaccines comprising the chimeric proteins for Helicobacter pylori-associated diseases.

2. Description of the Prior Art

Although gastritis-associated diseases such as gastritis, gastric ulcer and duodenal ulcer are caused by various etiological factors, they are mainly caused by Helicobacter pylori (hereinafter, referred to as ‘H. pylori’) colonizing in the junctional region of epithelial cells of stomach mucous membrane. It has been reported that 90% or more of Asians and 60% or more of Europeans are infected with H. pylori though there are local differences. Also, it has been known that recurrence of gastritis, gastric ulcer or duodenal ulcer is caused by drug-resistant H. pylori, which may give rise to the occurrence of gastric cancer (see: Timothy, et al., ASM News, 61:21(1995)).

So far, a variety of chemical therapeutic agents such as antibiotics and anti-ulcer agents have been used, in order to treat the gastritis-associated diseases caused by H. pylori. However, these drugs have revealed some drawbacks as followings: limitation in penetrating the mucous membrane of intestines, emergence of drug-resistant microorganisms, occurrence of reinfection and untoward effects of the drugs. Under the circumstances, there are strong reasons for exploring and developing alternative drugs for the control of H. pylori employing new therapeutic approach, e.g., immunological therapy which can substitute for chemical therapy.

Recently, in order to solve the said problems, studies on the development of vaccines to H. pylori have been carried out. As a result, diagnostic agents of H. pylori infection and preventive vaccines for H. pylori-associated diseases have been developed, employing genes coding for antigenic determinants of H. pylori, e.g., urease gene (see: Timothy, et al., Infection and Immunity, 59:1264(1991)), flagella gene (see: Leying, et al., Molecular Microbiology, 6:2863(1992)), adhesin gene (see: Evans, et al., Journal of Bacteriology, 175:674(1993)), superoxide dismutase gene (see: Christiane, et al., Infection and Immunity, 61:5315(1993)), catalase gene and vacuolating cytotoxin gene (see: Timothy, et al., Infection and Immunity, 58:603(1990)), some of which have been tested in preclinical phase.

However, they have not been manufactured up to now, owing to the following disadvantages: a vaccine employing an urease gene has poor immunogenicity; a vaccine employing a vacuolating cytotoxin gene may have toxicity of cytotoxin itself, though it provides excellent immunogenicity; a vaccine employing a non-toxic varient gene of the vacuolating cytotoxin gene does not have effects on all over the strains of H. pylori, since the non-toxic varient gene does not appear in all H. pylori; and, a vaccine employing adhesin gene, despite its excellent immunogenicity, does not have good efficacy, since it does not stimulate production of secretory IgA (“sIgA”).

In general, H. pylori is controlled by sIgA not by serum IgG, since it colonizes in the junctional region of epithelial cells of stomach mucous membrane. However, since the aforesaid vaccines cannot penetrate the mucous membrane of intestines easily, they are not able to stimulate mucosal immune system, which, in turn, results in decreased production of sIgA. Thus, serious problems have occurred that immunological effects of the vaccines against H. pylori decrease and the vaccines are easily denaturated by gastric acid (pH 1-2) to provide poor activities.

### SUMMARY OF THE INVENTION

Since vaccines employing H. pylori gene alone have the said various disadvantages, the present inventors have made an effort to prepare a chimeric protein fused with a protein which can penetrate mucous membrane of intestines easily and stimulate mucosal immune system to produce sIgA, as a fusion partner, for the purpose of using the chimeric protein as a potential vaccine for H. pylori.

Thus, the present inventors, first, prepared chimeric proteins expressed from recombinant DNAs which contain genes coding for antigenic determinants of H. pylori and A2 and B subunit genes of Vibro cholerae toxin. Also, they discovered that the chimeric proteins successfully solve the problems of the conventional vaccines and can be used as effective vaccines for H. pylori, based on their excellent immunogenicity for H. pylori, stability under stomach environment, and penetrating property through intestinal membrane to stimulate sIgA production.

The first object of the invention is, therefore, to provide a series of DNA sequences prepared by ligating antigenic determinant coding genes of H. pylori and A2 and B subunit genes of Vibro cholerae toxin, and amino acid sequences translated therefrom.

The second object of the invention is to provide expression vectors comprising the said DNA sequences and recombinant microorganisms transformed therewith.

The third object of the invention is to provide a process for preparing chimeric proteins consisting antigen proteins of H. pylori and A2 and B subunits of Vibro cholerae toxin from the said microorganisms.

The fourth object of the invention is to provide preventive and therapeutic vaccines for H. pylori-associated diseases employing the chimeric proteins prepared above.

### BRIEF DESCRIPTION OF THE DRAWINGS

The above and the other objects and features of the present invention will become apparent from the following descriptions given in conjunction with the accompanying drawings, in which:

FIG. 1 shows a DNA sequence of a fusion gene prepared by ligating ureB gene of H. pylori and A2 and B subunit genes of Vibro cholerae toxin.

FIG. 2 shows an amino acid sequence translated from the DNA sequence of FIG. 1.

FIG. 3 shows a DNA sequence of a fusion gene prepared by ligating cagA gene of H. pylori and A2 and B subunit genes of Vibro cholerae toxin.

FIG. 4 shows an amino acid sequence translated from the DNA sequence of FIG. 3.

FIG. 5 is a schematic diagram showing construction strategy of an expression vector for UreB/CTXA2B, pHU044.

FIG. 6 is a schematic diagram showing construction strategy of an expression vector for CagA/CTXA2B, pHC033.

FIG. 7 is a photograph showing 15% SDS-PAGE pattern of whole cell lysate of E. coli transformed with pHU044 expression vector.

FIG. 8 is a photograph showing 15% SDS-PAGE pattern of whole cell lysate of E. coli transformed with pHC033 expression vector.

FIG. 9 is a chromatogram showing comparison of serum IgG production of mice immunized with UreB/CTXA2B chimeric protein and UreB, respectively.

FIG. 10 is a chromatogram showing comparison of secretory IgA production of mice immunized with UreB/CTXA2B chimeric protein and UreB, respectively.

FIG. 11 is a chromatogram showing comparison of serum IgG production of mice immunized with CagA/CTXA2B chimeric protein and CagA, respectively.

FIG. 12 is a chromatogram showing comparison of secretory IgG production of mice immunized with CagA/CTXA2B chimeric protein and CagA, respectively.

### DETAILED DESCRIPTION OF THE INVENTION

The present inventors first gave an attention to the following characteristics of a toxin gene of Vibrio cholerae in the course of searching for a fusion partner of H. pylori gene: A gene of Vibro cholerae toxin consists of genes coding for three subunits of A1, A2 and B. A1 subunit has a toxic activity of the toxin, and A2 and B subunits bind to host cell to stimulate production of sIgA and guarantee stability of the protein under a surrounding environment. Also, vaccines employing A2 and B subunit genes of Vibrio cholerae toxin can be applied to human body, due to their tolerable characteristics, while various vaccines employing intact cholera toxin gene as a fusion partner, owing to toxic property of A1 subunit, can not be used directly for human body. Further, studies on a vaccine employing A2 and B subunits of cholera toxin as a fusion partner, have revealed that production of sIgA and serum IgG is stimulated when a chimeric protein prepared by ligating A2 and B subunit genes of cholera toxin with adhesin gene of Streptococcus mutans is orally administrated (see: Hajishengallis, et al., The Journal of Immunology, 154:4322(1995)).

Grounded on the afore-mentioned knowledges, the present inventors prepared chimeric proteins employing A2 and B subunit genes of Vibro cholerae toxin and antigenic determinant coding genes of H. pylori whose products have excellent immunogenicity, in order to stimulate production of antibody to H. pylori. The antigenic determinant coding genes of H. pylori include ureB, cagA, alpA, alpB, fliQ, babA1, babA2, ureC, ureD, ureA, sodB, ureI, ureE, ureF, ureG, ureH, flaA, flaB, catA, vacA, and babB.

First, the antigenic determinant coding genes of H. pylori and A2 and B subunit genes of Vibro cholerae toxin were prepared by employing polymerase chain reaction (PCR) technique, respectively. Then, each gene was cleaved with EcoRI and said two genes were ligated with T₄ DNA ligase. The fusion genes thus prepared were cleaved with restriction enzymes, and inserted into plasmid vectors to prepare respective recombinant expression vectors. Then, E. coli was transformed with each of expression vector, the recombinant E. coli was cultured, and chimeric proteins of antigenic proteins of H. pylori and A2 and B subunits of Vibrio cholerae toxin were obtained.

The antibody production rates and their effects as potential vaccines against H. pylori of the said chimeric proteins were examined. As a result, it was found that use of the said chimeric proteins permit to solve the problem of the conventional vaccines using the antigenic proteins, i.e., no stimulation of sIgA production, and mice immunized with the said chimeric proteins produced considerable amount of sIgA compared to mice administered with the antigenic protein alone. Also, it was revealed that mice immunized with the chimeric proteins showed prevention rate against infection with H. pylori of 75% while control group and mice immunized with only antigenic protein showed lower than 55% of prevention rate, respectively, which clearly demonstrates that the chimeric proteins may be used as active ingredients of vaccines for prevention and treatment of H. pylori-associated diseases, diagnostic kits for H. pylori infection, and used in the production of anti-H. pylori antibody.

The chimeric proteins of the invention induce mucosal immune response to bring about infiltration of IgA antibodies and/or lymphocytes into gastric mucosa. Thus, prevention of H. pylori infection or removal of H. pylori already infected can be accomplished. Accordingly, the chimeric proteins can be administered for the prevention of H. pylori infection of normal people or for the removal of H. pylori and the treatment of H. pylori-associated diseases of H. pylori-infected patients.

The chimeric proteins of the invention can be manufactured in a medicament for the conventional oral administration such as solutions, tablets, capsules and granules, and administered orally.

The said medicament for the oral administration can be manufactured by formulating them with pharmaceutically acceptable buffering agents such as sodium bicarbonate, potassium bicarbonate and sodium phosphate to protect the chimeric proteins stably by increasing pH of gastric juice or neutralizing the gastric juice, and manufactured by formulating them with various pharmaceutically acceptable carriers such as stabilizers and sweeteners.

Also, the medicament can be mixed with other antibiotics, etc. for effective prevention of H. pylori infection and removal of H. pylori, and with various anti-ulcer agents for shortening of period required for the treatment of gastritis, gastric ulcer or duodenal ulcer.

In general, the chimeric proteins, in case of an adult of 60 kg body weight, may be administered preferably in one dose of 10 μg to 1,000 mg per day, and the dosage may be changed by the conventionally skilled in the art. If necessary, re-administration may be performed at 1-week or 2-week intervals to induce a booster reaction, and a booster dose may be the same as or lower than that during the primary administration.

As a result of oral administration of the chimeric proteins into 10 mice, it was found that all of the proteins have LD₅₀ of 4 g/kg or more, which shows that the chimeric proteins are sufficiently safe in the range of effective dose.

The present invention further provides preventive and therapeutic vaccines for H. pyroli-associated diseases which comprise the chemeric proteins' functional equivalents.

In describing the amino acid sequence and the nucleotide sequence of the present invention, the term ‘functional equivalents’ is employed to mean all proteins substituted by the combinations such as Gly, Ala; Val, Ile, Leu; Asp, Glu; Asn, Gln; Ser, Thr; Lys, Arg; and, Phe, Tyr among the amino acid sequences of the chimeric protein, and all genes comprising nucleotide sequences coding for all the said combinations among the nucleotide sequences of the fusion gene, respectively.

The present invention is further illustrated in the following examples, which should not be taken to limit the scope of the invention.

### EXAMPLE 1

### Isolation of Chromosomal DNA from H. pylori

H. pylori 11637 RPH 13487(ATCC 43504) was cultured in the BHI (brain heart infusion) liquid medium (consisting of 10 mg/ml vancomycin, 5 mg/ml trimetofrim and 4 mg/ml amphotericin B) containing 5% horse serum, and incubated for 72 hours under an environment of 10% (v/v) CO₂. Then, chromosomal DNA was isolated from the cultured cells by the conventional method in the art.

### EXAMPLE 2

### Synthesis of Oligonucleotides for Amplification of Antigenic Determinant Coding Genes

### EXAMPLE 2-1

Synthesis of Oligonucleotides for ureB Gene Amplification

Two oligonucleotides of 37-mer and 30-mer as followings, were synthesized to amplify ureB gene of H. pylori by PCR technique described in Example 3 below:

5′-CCGTG GATGA AAAAG ATTAG CAGAA AAGAA TATGC TT-3′

5′-AGAAT TCTCA CTTTA TTGGC TGGTT TAGAG-3′

In an analogous manner, two oligonucleotides of 28-mer and 27-mer as followings, were synthesized to amplify A2 and B subunit genes of Vibro cholerae toxin:

5′-AGAAT TCGAA GAGCC GTGGA TTCAT CAT-3′

5′-ACTGC AGCAC ATAAT ACGCA CTAAG GA-3′

In this connection, the oligonucleotides were synthesized employing an automatic nucleotide synthesizer (Pharmacia-LKB Biotechnology, Uppsala, Sweden).

The oligonucleotides thus prepared were reacted with TTD (thiophenol/triethylamine/dioxane=1/2/2, v/v/v) solution in a silica matrix, washed with methanol and ethanol sufficiently, and treated with strong ammonia water to separate the synthesized oligonucleotides from the silica matrix. To the oligonucleotide solutions thus obtained was further added strong ammonia water. Then, the solutions were left to stand at 50° C. for 12 hours, and concentrated under a reduced pressure with gas removal to reach a final volume of 0.5 ml. And then, using the oligonucleotides thus concentrated, primary purification was carried out with acetonitrile/triethylamine buffer employing a SEP-PAK cartridge (Waters Inc., USA), and electrophoresis was performed using 15% polyacrylamide gel (in TE-borate, pH 8.3). After electrophoresis, oligonucleotides were visualized under shortwave ultraviolet rays, and only the gel fragments corresponding to the oligonucleotides were cleaved. Then, oligonucleotides were electroeluted from the gel fragments, while remaining salts with acetonitrile/triethylamine buffer employing SEP-PAK cartridge connected with a syringe to purify each oligonucleotide. Oligonucleotides thus purified were labelled with γ-[³²P]-ATP employing T₄ polynucleotide kinase (New England Biolabs, #201S, USA) and the nucleotide sequences were determined by Maxam and Gilbert's nucleotide sequencing method (see: Maxam, A. M. & Gilbert, W., Proc. Natl. Acad. Sci., USA, 74:560-564(1977))

### EXAMPLE 2-2

### Synthesis of Oligonucleotides for cagA Gene Amplification

Two oligonucleotides of 37-mer and 30-mer as followings, were synthesized to amplify cagA gene of H. pylori in an analogous manner as in Example 2-1:

5′-CCGTG GATGA CTAAC GAAAC CATTG ACCAA CAACC AC-3′

5′-AGAAT TCTTA AGATT TTTGG AAACC ACCTT-3′

### EXAMPLE 2-3

### Synthesis of Oligonucleotides for alpA Gene Amplification

Two oligonucleotides of 23-mer and 21-mer as followings, were synthesized to amplify alpA gene of H. pylori in an analogous manner as in Example 2-1:

5′-CCGTG GATGA TAAAA AAGAA TAG-3′

5′-GAATT CTTAG AATGA ATACC C-3′

### EXAMPLE 2-4

### Synthesis of Oligonucleotides for alpB Gene Amplification

Two oligonucleotides of 25-mer and 21-mer as followings, were synthesized to amplify alpB gene of H. pylori in an analogous manner as in Example 2-1:

5′-CCGTG GATGA CACAA TCTCA AAAAG-3′

5′-GAATT CTTAG AAGGC GTAGC C-3′

### EXAMPLE 2-5

### Synthesis of Oligonucleotides for fliQ Gene Amplification

Two oligonucleotides of 24-mer and 21-mer as followings, were synthesized to amplify fliQ gene of H. pylori in an analogous manner as in Example 2-1:

5′-CCGTG GATGG AATCA CAACT CATG-3′

5′-GAATT CGCCT ATGAT TTTGG G-3′

### EXAMPLE 2-6

### Synthesis of Oligonucleotides for babA1 Gene Amplification

Two oligonucleotides of 21-mer and 21-mer as followings, were synthesized to amplify babA1 gene of H. pylori in an analogous manner as in Example 2-1:

5′-CCGTG GATGG TAACA AACAC C-3′

5′-GAATT CTTAG TAAGC GAACA C-3′

### EXAMPLE 2-7

### Synthesis of Oligonucleotides for babA2 Gene Amplification

Two oligonucleotides of 22-mer and 21-mer as followings, were synthesized to amplify babA2 gene of H. pylori in an analogous manner as in Example 2-1:

5′-CCGTG GATGA AAAAA CACAT CC-3′

5′-GAATT CTTAA TAAGC GAACA C-3′

### EXAMPLE 2-8

### Synthesis of Oligonucleotides for ureC Gene Amplification

Two oligonucleotides of 21-mer and 23-mer as followings, were synthesized to amplify ureC gene of H. pylori in an analogous manner as in Example 2-1:

5′-CCGTG GATGA AAATT TTTGG G-3′

5′-GAATT CTTAG CACAA ATGCC CTT-3′

### EXAMPLE 2-9

### Synthesis of Oligonucleotides for ureD Gene Amplification

Two oligonucleotides of 24-mer and 23-mer as followings, were synthesized to amplify ureD gene of H. pylori in an analogous manner as in Example 2-1:

5′-CCGTG GGTGC TAAAA ACCAC TAAA-3′

5′-GAATT CTCAT GACAT CAGCG AAG-3′

### EXAMPLE 2-10

### Synthesis of Oligonucleotides for ureA Gene Amplification

Two oligonucleotides of 25-mer and 22-mer as followings, were synthesized to amplify ureA gene of H. pylori in an analogous manner as in Example 2-1:

5′-CCGTG GATGA AACTC ACCCC AAAAG-3′

5′-GAATT CTTAC TCCTT AATTG TT-3′

### EXAMPLE 2-11

### Synthesis of Oligonucleotides for sodB Gene Amplification

Two oligonucleotides of 24-mer and 25-mer as followings, were synthesized to amplify sodB gene of H. pylori in an analogous manner as in Example 2-1:

5′-CCGTG GATGT TTACA TTACG AGAG-3′

5′-GAATT CTCAT TCAAG CTTTT TATGC-3′

### EXAMPLE 2-12

### Synthesis of Oligonucleotides for ureI Gene Amplification

Two oligonucleotides of 26-mer and 24-mer as followings, were synthesized to amplify ureI gene of H. pylori in an analogous manner as in Example 2-1:

5′-CCGTG GATGC TAGGA CTTGT ATTGT T-3′

5′-GAATT CTCAC ACCCA GTGTT GGAT-3′

### EXAMPLE 2-13

### Synthesis of Oligonucleotides for ureE Gene Amplification

Two oligonucleotides of 22-mer and 21-mer as followings, were synthesized to amplify ureE gene of H. pylori in an analogous manner as in Example 2-1:

5′-CCGTG GATGA TCATA GAGCG TT-3′

5′-GAATT CCTAT TTCAT GACCA C-3′

### EXAMPLE 2-14

### Synthesis of Oligonucleotides for ureF Gene Amplification

Two oligonucleotides of 25-mer and 23-mer as followings, were synthesized to amplify ureF gene of H. pylori in an analogous manner as in Example 2-1:

5′-CCGTG GATGG ATAAA GGAAA AAGCG-3′

5′-GAATT CTCAA GACAT ATAAA GGC-3′

### EXAMPLE 2-15

### Synthesis of Oligonucleotides for ureG Gene Amplification

Two oligonucleotides of 25-mer and 25-mer as followings, were synthesized to amplify ureG gene of H. pylori in an analogous manner as in Example 2-1:

5′-CCGTG GATGG TAAAA ATTGG AGTTT-3′

5′-GAATT CTCAA TCTTC CAATA AAGCG-3′

### EXAMPLE 2-16

### Synthesis of Oligonucleotides for ureH Gene Amplification

Two oligonucleotides of 22-mer and 20-mer as followings, were synthesized to amplify ureH gene of H. pylori in an analogous manner as in Example 2-1:

5′ CCGTG GATGA ACACT TACGC TC-3′

5′ GAATT CTTAA ACCTT TGGCG-3′

### EXAMPLE 2-17

### Synthesis of Oligonucleotides for flaA Gene Amplification

Two oligonucleotides of 21-mer and 20-mer as followings, were synthesized to amplify flaA gene of H. pylori in an analogous manner as in Example 2-1:

5′-CCGTG GATGG CTTTT CAGGT C-3′

5′-GAATT CCTAA GTTAA AAGCC-3′

### EXAMPLE 2-18

### Synthesis of Oligonucleotides for flaB Gene Amplification

Two oligonucleotides of 23-mer and 21-mer as followings, were synthesized to amplify flaB gene of H. pylori in an analogous manner as in Example 2-1:

5′-CCGTG GATGA GTTTT AGGAT AAA-3′

5′-GAATT CTTAT TGTAA AAGCC T-3′

### EXAMPLE 2-19

### Synthesis of Oligonucleotides for catA Gene Amplification

Two oligonucleotides of 24-mer and 27-mer as followings, were synthesized to amplify catA gene of H. pylori in an analogous manner as in Example 2-1:

5′-CCGTG GATGG TTAAT AAAGA TGTG-3′

5′-GAATT CTTAC TTTTT CTTTT TTGTG TG-3′

### EXAMPLE 2-20

### Synthesis of Oligonucleotides for vacA Gene Amplification

Two oligonucleotides of 24-mer and 26-mer as followings, were synthesized to amplify vacA gene of H. pylori in an analogous manner as in Example 2-1:

5′-CCGTG GGCCT TTTTT ACAAC CGTG-3′

5′-GAATT CTTAG AAACT ATACC TCAGG C-3′

### EXAMPLE 2-21

### Synthesis of Oligonucleotides for babB Gene Amplification

Two oligonucleotides of 23-mer and 21-mer as followings, were synthesized to amplify babB gene of H. pylori in an analogous manner as in Example 2-1:

5′-CCGTG GATGA AAAAA AACCC TTT-3′

5′-GAATT CCTAG TAAGC GAACA C-3′

### EXAMPLE 3

### Amplification of Antigenic Determinant Genes

### EXAMPLE 3-1

Amplification of ureB Gene and A2/B Subunit Genes of Vibrio cholerae

To the solution containing template DNA (10 ng), 10 μl of 10× Taq polymerase buffer (10 mM Tris-HCl (pH 8.3) containing 500 mM KCl, 15 mM MgCl₂ and 0.1% (v/v) gelatin), 10 μl of dNTP's mixture (containing an equimolar concentration of 1.25 mM dGTP, dATP, dTTP and dCTP), 2 μg of each primer (oligonucleotides synthesized in Example 2-1) and 0.5 μl of Ampli Taq DNA polymerase (Perkin-Elmer Cetus, USA), was added distilled water to be a final volume of 100 μl. In order to prevent evaporation of the solution, 50 μl of mineral oil was added to the solution. In case of amplification of ureB gene of H. Pylori, chromosomal DNA of H. pylori isolated in Example 1 was used as a template DNA, and oligonucleotides synthesized in Example 2-1, i.e., 37-mer and 30-mer, were used as primers; and, in case of amplification of A2 and B subunit genes of Vibro cholerae toxin, chromosomal DNA of Vibrio cholerae was used as a template DNA, and oligonucleotides synthesized in Example 2-1, i.e. , 28-mer and 27-mer, were used as primers.

Denaturation (95° C., 1 minute), annealing (55° C., 1 minute), and extension (72° C., 2 minute) were carried out for 30 cycles in a serial manner, using Thermal Cycler™ (Cetus/Perkin-Elmer, USA), and final reaction was carried out at 72° C. for 10 minutes. And then, in order to remove polymerase, the equal volume of phenol/chloroform mixture (1:1 (v/v)) was added to the reaction mixture, mixed well, and subsequently centrifuged. The supernatant thus obtained was transferred to a fresh tube. Then, 1/10 volume of 3M sodium acetate and 2 volume of 100% ethanol was added to the supernatant, mixed and centrifuged to obtain double-stranded nucleic acid. The nucleic acid was dissolved in 20 μl of TE buffer for later use.

### EXAMPLE 3-2

### Amplification of cagA Gene and A2/B Subunit Genes of Vibrio cholerae

To the solution containing template DNA (10 ng), 10 μl of 10× Taq polymerase buffer (10 mM Tris-HCl (pH 8.3) containing 500 mM KCl, 15 mM MgCl₂ and 0.1% (v/v) gelatin), 10 μl of dNTP's mixture (containing an equimolar concentration of 1.25 mM dGTP, dATP, dTTP and dCTP), 2 μg of each primer (oligonucleotides synthesized in Example 2-2) and 0.5 μl of Ampli Taq DNA polymerase (Perkin-Elmer Cetus, USA), was added distilled water to be a final volume of 100 μl. In order to prevent evaporation of the solution, 50 μl of mineral oil was added to the solution. In case of amplification of cagA gene of H. pylori, chromosomal DNA of H. pylori isolated in Example 1 was used as a template DNA, and oligonucleotides synthesized in Example 2-2, i.e., 37-mer and 30-mer, were used as primers; and, in case of amplification of A2 and B subunit genes of Vibro cholerae toxin, chromosomal DNA of Vibrio cholerae was used as a template DNA, and oligonucleotides synthesized in Example 2-1, i.e., 28-mer and 27-mer, were used as primers.

Denaturation (95° C., 1 minute), annealing (55° C., 1 minute), and extension (72° C., 2 minute) were carried out for 30 cycles in a serial manner, using Thermal Cycler™ (Cetus/Perkin-Elmer, USA), and final reaction was carried out at 72° C. for 10 minutes. And then, in order to remove polymerase, the equal volume of phenol/chloroform mixture (1:1 (v/v)) was added to the reaction mixture, mixed well, and subsequently centrifuged. The supernatant thus obtained was transferred to a fresh tube. Then, 1/10 volume of 3M sodium acetate and 2 volume of 100% ethanol was added to the supernatant, mixed and centrifuged to obtain double-stranded nucleic acid. The nucleic acid was dissolved in 20 μl of TE buffer for later use.

### EXAMPLE 3-3

### Amplification of alpA Gene and A2/B Subunit Genes of Vibrio cholerae

alpA gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 23-mer and 21-mer synthesized in Example 2-3 were employed as primer.

### EXAMPLE 3-4

### Amplification of alpB gene and A2/B Subunit Genes of Vibrio cholerae

alpB gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 25-mer and 21-mer synthesized in Example 2-4 were employed as primer.

### EXAMPLE 3-5

### Amplification of fliQ Gene and A2/B Subunit Genes of Vibrio cholerae

fliQ gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 24-mer and 21-mer synthesized in Example 2-5 were employed as primer.

### EXAMPLE 3-6

### Amplification of babA1 gene and A2/B Subunit Genes of Vibrio cholerae

babA1 gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 21-mer and 21-mer synthesized in Example 2-6 were employed as primer.

### EXAMPLE 3-7

### Amplification of babA2 Gene and A2/B Subunit Genes of Vibrio cholerae

babA2 gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 22-mer and 21-mer synthesized in Example 2-7 were employed as primer.

### EXAMPLE 3-8

### Amplification of ureC Gene and A2/B Subunit Genes of Vibrio cholerae

ureC gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 21-mer and 23-mer synthesized in Example 2-8 were employed as primer.

### EXAMPLE 3-9

### Amplification of ureD Gene and A2/B Subunit Genes of Vibrio cholerae

ureD gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 24-mer and 23-mer synthesized in Example 2-9 were employed as primer.

### EXAMPLE 3-10

### Amplification of ureA Gene and A2/B Subunit Genes of Vibrio cholerae

ureA gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 25-mer and 22-mer synthesized in Example 2-10were employed as primer.

### EXAMPLE 3-11

### Amplification of sodB Gene and A2/B Subunit Genes of Vibrio cholerae

sodB gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 24-mer and 25-mer synthesized in Example 2-11 were employed as primer.

### EXAMPLE 3-12

### Amplification of ureI Gene and A2/B Subunit Genes of Vibrio cholerae

ureI gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 26-mer and 24-mer synthesized in Example 2-12 were employed as primer.

### EXAMPLE 3-13

### Amplification of ureE Gene and A2/B Subunit Genes of Vibrio cholerae

ureE gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 22-mer and 21-mer synthesized in Example 2-13were employed as primer.

### EXAMPLE 3-14

### Amplification of ureF Gene and A2/B Subunit Genes of Vibrio cholerae

ureF gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 25-mer and 23-mer synthesized in Example 2-14 were employed as primer.

### EXAMPLE 3-15

### Amplification of ureG Gene and A2/B Subunit Genes of Vibrio cholerae

ureG gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 25-mer and 25-mer synthesized in Example 2-15 were employed as primer.

### EXAMPLE 3-16

### Amplification of ureH Gene and A2/B Subunit Genes of Vibrio cholerae

ureH gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 22-mer and 20-mer synthesized in Example 2-16 were employed as primer.

### EXAMPLE 3-17

### Amplification of flaA Gene and A2/B Subunit Genes of Vibrio cholerae

flaA gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 21-mer and 20-mer synthesized in Example 2-17 were employed as primer.

### EXAMPLE 3-18

### Amplification of flaB Gene and A2/B Subunit Genes of Vibrio cholerae

flaB gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 23-mer and 21-mer synthesized in Example 2-18 were employed as primer.

### EXAMPLE 3-19

### Amplification of catA Gene and A2/B Subunit Genes of Vibrio cholerae

catA gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 24-mer and 27-mer synthesized in Example 2-19 were employed as primer.

### EXAMPLE 3-20

### Amplification of vacA Gene and A2/B Subunit Genes of Vibrio cholerae

vacA gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 24-mer and 26-mer synthesized in Example 2-20 were employed as primer.

### EXAMPLE 3-21

### Amplification of babB Gene and A2/B Subunit Genes of Vibrio cholerae

babB gene and A2/B subunit gene of Vibro cholerae were amplified in an analogus manner as in Example 3-1, with the exceptions that: chromosomal DNA of H. pylori isolated in Example 1 was employed as template DNA, and oligonucleotides of 23-mer and 21-mer synthesized in Example 2-21 were employed as primer.

### EXAMPLE 4

### Construction of Expression Vectors Expressing Chimeric Proteins

### EXAMPLE 4-1

Construction of an Expression Vector, pHU044

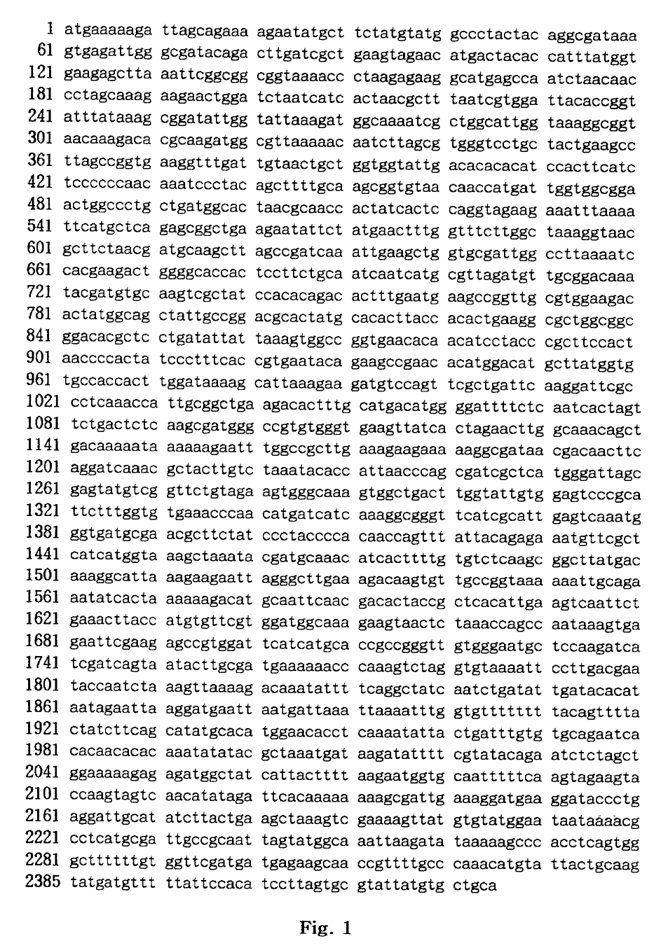

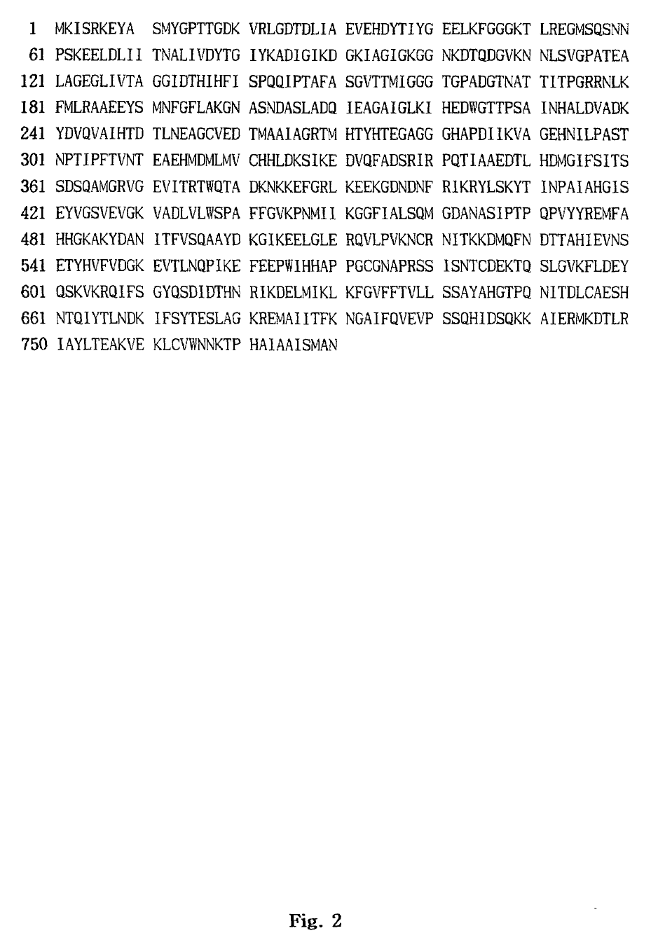

The ureB gene of H. pylori and the A2 and B subunit genes of Vibro cholerae toxin amplified in Example 3-1, respectively, were digested with EcoRI, respectively. Each of 1 μg of H. pylori DNA and Vibro cholerae DNA was mixed. Then, 3 μl of 10× concentrated solution for fusion (600 mM Tris-HCl buffer (pH 7.5) containing 10 mM DTT and 100 mM MgCl₂), 1 μl of 10 mM ATP and 10 unit of T₄ DNA ligase were added to the DNA mixture to reach a final reaction volume of 30 μl, and held at 14° C. for 16 hours. After 1% agarose gel electrophoresis of the reaction product, a fusion gene of about 2.4 kb was obtained and its nucleotide sequence was determined (see: FIG. 1). In FIG. 1, nucleotide sequence of base position 1 to 1679 corresponds to signal peptide sequence of the ureB, and nucleotide sequence of base position 1680 to 1712 corresponds to signal peptide sequence of the B subunit of Vibro cholerae toxin. FIG. 2 shows an amino acid sequence translated from the DNA sequence of FIG. 1.

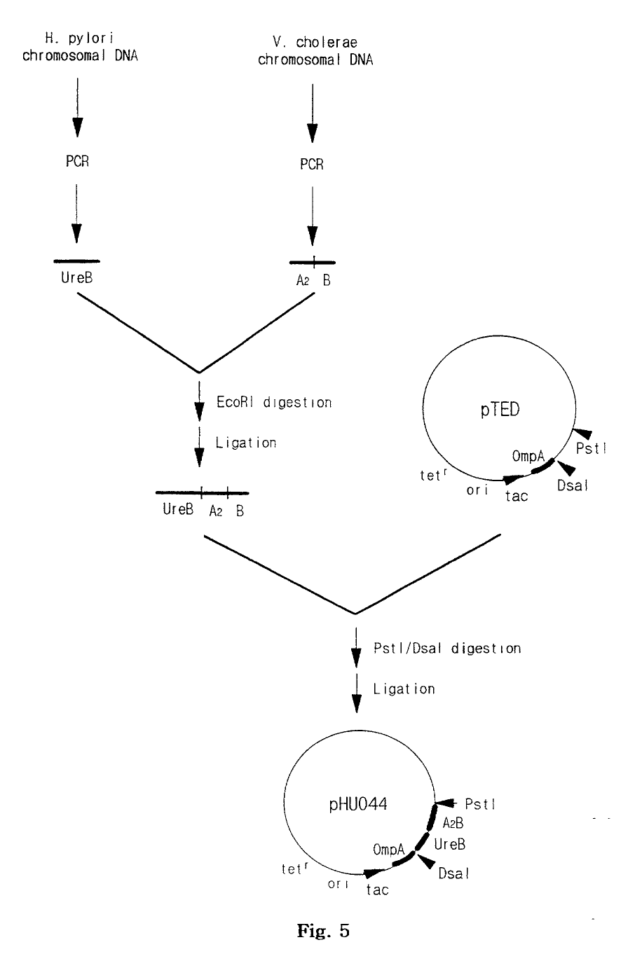

The fusion gene having the nucleotide sequence thus determined was double-digested with DsaI and PstI, and inserted into pTED plasmid vector double-digested with the said restriction enzymes to prepare a circular plasmid which was designated as ‘pHU044’. The said plasmid pTED is 2.95 kb plasmid which was created DsaI restriction enzyme recognition site to pTE105, isolated from E. coli JM 101 (DW/BT-2042)transformed with pTE105 (KCCM-10027). FIG. 5 is a schematic diagram showing the construction strategy of pHU044.

Further, treatment of pHU044 with restriction enzyme and 1% agarose gel electrophoresis revealed that: the pHU044 expression vector has unique restriction site for each restriction enzyme; and the fusion gene was correctly inserted.

### EXAMPLE 4-2

### Construction of an Expression Vector, pHC033

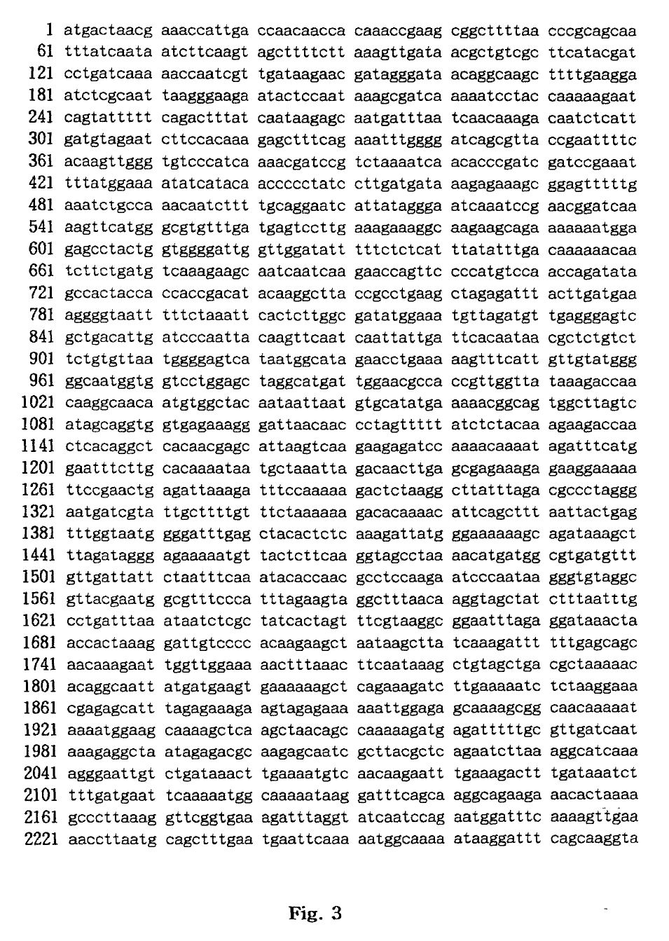

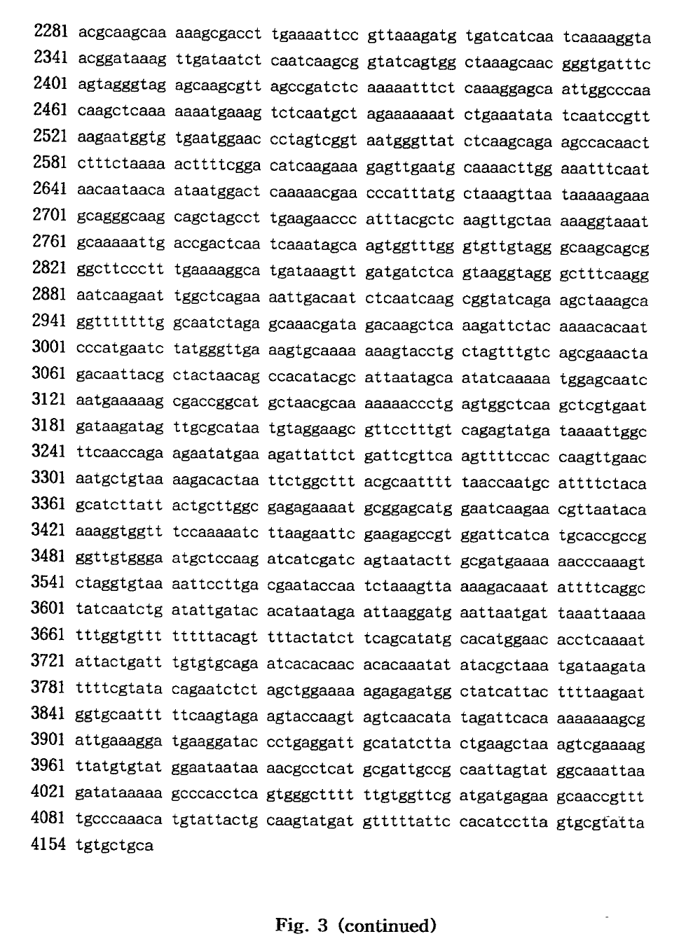

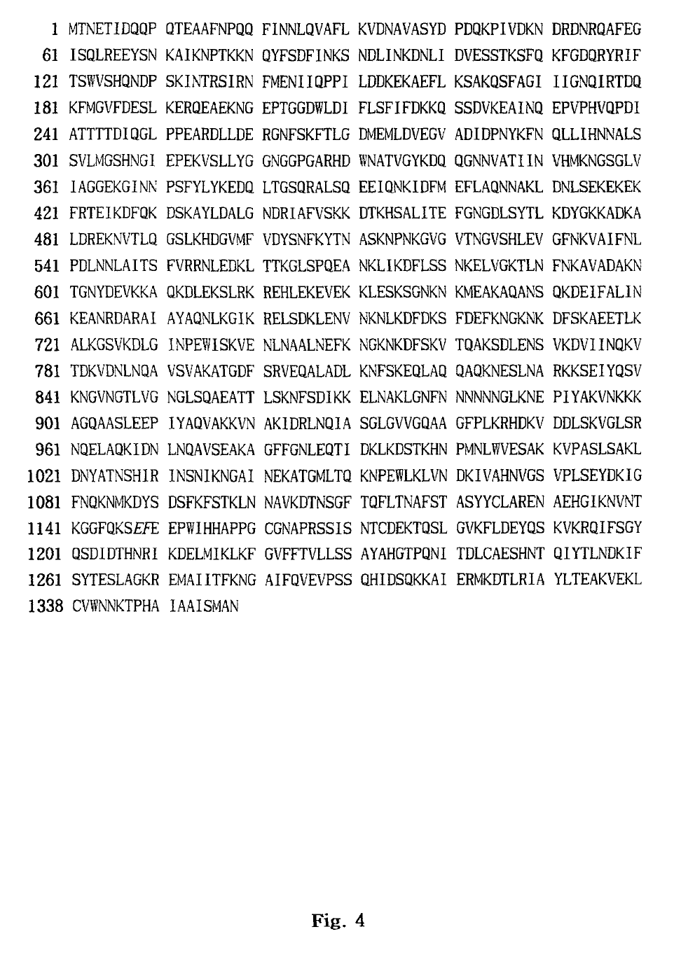

The cagA gene of H. pylori and the A2 and B subunit genes of Vibro cholerae toxin amplified in Example 3-2, respectively, were digested with EcoRI, respectively. Each of 1 μg of H. pylori DNA and Vibro cholerae DNA was mixed. Then, 3 μl of 10× concentrated solution for fusion (600 mM Tris-HCl buffer (pH 7.5) containing 10 mM DTT and 100 mM MgCl₂), 1 μl of 10 mM ATP and 10 unit of T₄ DNA ligase were added to the DNA mixture to reach a final reaction volume of 30 μl, and held at 14° C. for 16 hours. After 1% agarose gel electrophoresis of the reaction product, a fusion gene of about 4.1 kb was obtained and its nucleotide sequence was determined (see: FIG. 3). In FIG. 3, nucleotide sequence of base position 1 to 3444 corresponds to signal peptide sequence of the cagA, and nucleotide sequence of base position 3445 to 3477 corresponds to signal peptide sequence of the B subunit of Vibro cholerae toxin. FIG. 4 shows an amino acid sequence translated from the DNA sequence of FIG. 3.

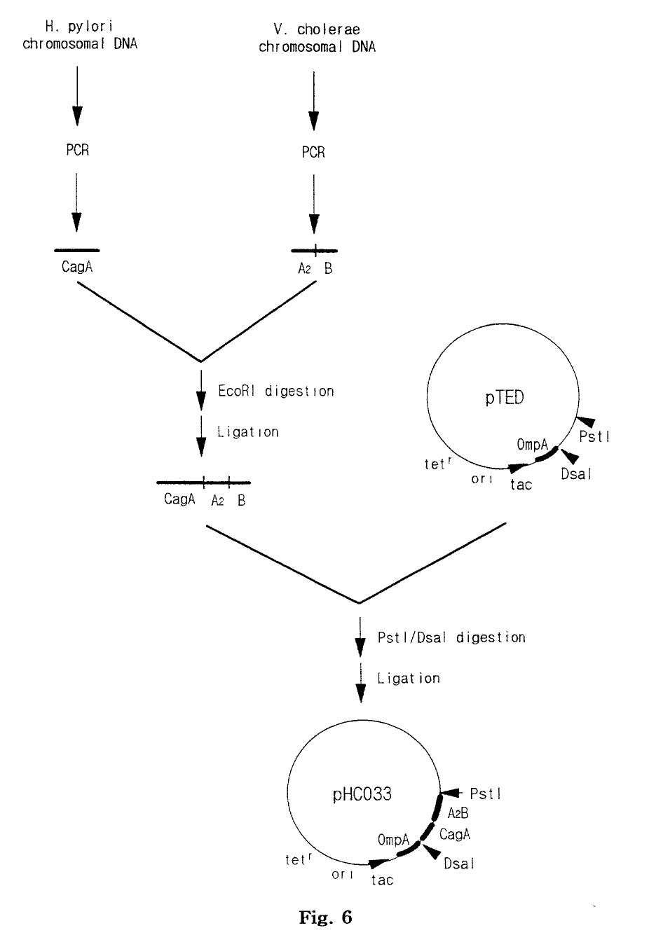

The fusion gene having the nucleotide sequence thus determined was double-digested with DsaI and PstI, and inserted into pTED plasmid vector double-digested with the said restriction enzymes to prepare a circular plasmid which was designated as ‘pHC033’. The said plasmid pTED is 2.95 kb plasmid which was created DsaI restriction enzyme recognition site to pTE105 isolated from E. coli JM101 (DW/BT-2042) transformed with pTE105(KCCM-10027). FIG. 6 is a schematic diagram showing the construction strategy of pHC033.

Further, treatment pHC033 with restriction enzyme and 1% agarose gel electrophoresis revealed that: the pHC033 expression vector has unique restriction site for each restriction enzyme; and the fusion gene was correctly inserted.

### EXAMPLE 4-3

### Construction of an Expression Vector Containing alpA Gene

The alpA gene of H. pylori and the A2 and B subunit genes of Vibro cholerae toxin amplified in Example 3-3, respectively, were digested with EcoRI, respectively. Each of 1 μg of H. pylori DNA and Vibro cholerae DNA was mixed. Then, 3 μg of 10× concentrated solution for fusion (600 mM Tris-HCl buffer (pH 7.5) containing 10 mM DTT and 100 mM MgCl₂), 1 μl of 10 mM ATP and 10 unit of T₄ DNA ligase were added to the DNA mixture to reach a final reaction volume of 30 μl, and held at 14° C. for 16 hours. After 1% agarose gel electrophoresis of the reaction product, a fusion gene of about 2.4 kb was obtained and its nucleotide sequence was determined. The fusion gene having the nucleotide sequence thus determined was double-digested with DsaI and PstI, and inserted into pTED plasmid vector double-digested with the said restriction enzymes to prepare an expression vector containing a chimeric gene of alpA gene and A2/B subunit gene of Vibro cholerae toxin.

### EXAMPLE 4-4

### Construction of an Expression Vector Containing alpB Gene

Expression vector containing a chimeric gene of alpB gene and A2/B subunit gene of Vibro cholerae toxin was prepared in an analogous manner as in Example 4-3, after sequence determination of the fused gene of alpB gene and A2/B subunit gene of Vibro cholerae toxin of 2.3 kb, with an exception of employing alpB gene of H. pylori amplified in

### EXAMPLE 3-4.

### EXAMPLE 4-5

### Construction of an Expression Vector Containing fliQ Gene

Expression vector containing a chimeric gene of fliQ gene and A2/B subunit gene of Vibro cholerae toxin was prepared in an analogous manner as in Example 4-3, after sequence determination of the fused gene of fliQ gene and A2/B subunit gene of Vibro cholerae toxin of 0.9 kb, with an exception of employing fliQ gene of H. pylori amplified in Example 3-5.

### EXAMPLE 4-6

### Construction of an Expression Vector Containing babA1 Gene

Expression vector containing a chimeric gene of babA1 gene and A2/B subunit gene of Vibro cholerae toxin was prepared in an analogous manner as in Example 4-3, after sequence determination of the fused gene of babA1 gene and A2/B subunit gene of Vibro cholerae toxin of 2.8 kb, with an exception of employing babA1 gene of H. pylori amplified in Example 3-6.

### EXAMPLE 4-7

### Construction of an Expression Vector Containing babA2 Gene

Expression vector containing a chimeric gene of babA2 gene and A2/B subunit gene of Vibro cholerae toxin was prepared in an analogous manner as in Example 4-3, after sequence determination of the fused gene of babA2 gene and A2/B subunit gene of Vibro cholerae toxin of 2.9 kb, with an exception of employing babA2 gene of H. pylori amplified in Example 3-7.

### EXAMPLE 4-8

### Construction of an Expression Vector Containing ureC Gene

Expression vector containing a chimeric gene of ureC gene and A2/B subunit gene of Vibro cholerae toxin was prepared in an analogous manner as in Example 4-3, after sequence determination of the fused gene of ureC gene and A2/B subunit gene of Vibro cholerae toxin of 2.0 kb, with an exception of employing ureC gene of H. pylori amplified in Example 3-8.

### EXAMPLE 4-9

### Construction of an Expression Vector Containing ureD Gene

Expression vector containing a chimeric gene of ureD gene and A2/B subunit gene of Vibro cholerae toxin was prepared in an analogous manner as in Example 4-3, after sequence determination of the fused gene of ureD gene and A2/B subunit gene of Vibro cholerae toxin of 1.1 kb, with an exception of employing ureD gene of H. pylori amplified in Example 3-9.

### EXAMPLE 4-10

### Construction of an Expression Vector Containing ureA Gene

Expression vector containing a chimeric gene of ureA gene and A2/B subunit gene of Vibro cholerae toxin was prepared in an analogous manner as in Example 4-3, after sequence determination of the fused gene of ureA gene and A2/B subunit gene of Vibro cholerae toxin of 1.4 kb, with an exception of employing ureA gene of H. pylori amplified in Example 3-10.

### EXAMPLE 4-11

### Construction of an Expression Vector Containing sodB Gene

Expression vector containing a chimeric gene of sodB gene and A2/B subunit gene of Vibro cholerae toxin was prepared in an analogous manner as in Example 4-3, after sequence determination of the fused gene of sodB gene and A2/B subunit gene of Vibro cholerae toxin of 1.3 kb, with an exception of employing sodB gene of H. pylori amplified in Example 3-11.

### EXAMPLE 4-12

### Construction of an Expression Vector Containing ureI Gene

Expression vector containing a chimeric gene of ureI gene and A2/B subunit gene of Vibro cholerae toxin was prepared in an analogous manner as in Example 4-3, after sequence determination of the fused gene of ureI gene and A2/B subunit gene of Vibro cholerae toxin of 1.3 kb, with an exception of employing ureI gene of H. pylori amplified in Example 3-12.

### EXAMPLE 4-13

### Construction of an Expression Vector Containing ureE Gene

Expression vector containing a chimeric gene of ureE gene and A2/B subunit gene of Vibro cholerae toxin was prepared in an analogous manner as in Example 4-3, after sequence determination of the fused gene of ureE gene and A2/B subunit gene of Vibro cholerae toxin of 1.2 kb, with an exception of employing ureE gene of H. pylori amplified in Example 3-13.

### EXAMPLE 4-14

### Construction of an Expression Vector Containing ureF Gene

Expression vector containing a chimeric gene of ureF gene and A2/B subunit gene of Vibro cholerae toxin was prepared in an analogous manner as in Example 4-3, after sequence determination of the fused gene of ureF gene and A2/B subunit gene of Vibro cholerae toxin of 1.5 kb, with an exception of employing ureF gene of H. pylori amplified in Example 3-14.

### EXAMPLE 4-15

### Construction of an Expression Vector Containing ureG Gene

Expression vector containing a chimeric gene of ureG gene and A2/B subunit gene of Vibro cholerae toxin was prepared in an analogous manner as in Example 4-3, after sequence determination of the fused gene of ureG gene and A2/B subunit gene of Vibro cholerae toxin of 1.3 kb, with an exception of employing ureG gene of H. pylori amplified in Example 3-15.

### EXAMPLE 4-16

### Construction of an Expression Vector Containing ureH Gene

Expression vector containing a chimeric gene of ureH gene and A2/B subunit gene of Vibro cholerae toxin was prepared in an analogous manner as in Example 4-3, after sequence determination of the fused gene of ureH gene and A2/B subunit gene of Vibro cholerae toxin of 1.5 kb, with an exception of employing ureH gene of H. pylori amplified in Example 3-16.

### EXAMPLE 4-17

### Construction of an Expression Vector Containing flaA Gene

Expression vector containing a chimeric gene of flaA gene and A2/B subunit gene of Vibro cholerae toxin was prepared in an analogous manner as in Example 4-3, after sequence determination of the fused gene of flaA gene and A2/B subunit gene of Vibro cholerae toxin of 2.2 kb, with an exception of employing flaA gene of H. pylori amplified in Example 3-17.

### EXAMPLE 4-18

### Construction of an Expression Vector Containing flaB Gene

Expression vector containing a chimeric gene of flaB gene and A2/B subunit gene of Vibro cholerae toxin was prepared in an analogous manner as in Example 4-3, after sequence determination of the fused gene of flaB gene and A2/B subunit gene of Vibro cholerae toxin of 2.2 kb, with an exception of employing flaB gene of H. pylori amplified in Example 3-18.

### EXAMPLE 4-19

### Construction of an Expression Vector Containing catA Gene

Expression vector containing a chimeric gene of catA gene and A2/B subunit gene of Vibro cholerae toxin was prepared in an analogous manner as in Example 4-3, after sequence determination of the fused gene of catA gene and A2/B subunit gene of Vibro cholerae toxin of 2.2 kb, with an exception of employing catA gene of H. pylori amplified in Example 3-19.

### EXAMPLE 4-20

### Construction of an Expression Vector Containing vacA Gene

Expression vector containing a chimeric gene of vacA gene and A2/B subunit gene of Vibro cholerae toxin was prepared in an analogous manner as in Example 4-3, after sequence determination of the fused gene of vacA gene and A2/B subunit gene of Vibro cholerae toxin of 4.5 kb, with an exception of employing vacA gene of H. pylori amplified in Example 3-20.

### EXAMPLE 4-21

### Construction of an Expression Vector Containing ureF Gene

Expression vector containing a chimeric gene of babB gene and A2/B subunit gene of Vibro cholerae toxin was prepared in an analogous manner as in Example 4-3, after sequence determination of the fused gene of babB gene and A2/B subunit gene of Vibro cholerae toxin of 1.4 kb, with an exception of employing babE gene of H. pylori amplified in Example 3-21.

### EXAMPLE 5

### Preparation of Transformants Expressing Chimeric Proteins

### EXAMPLE 5-1

### Preparation of a Transformant Containing pHU044

In order to transform host cell with the pHU044 expression vector, E. coli JM101 was first inoculated in liquid LB medium, cultured at 37° C. until absorbance at 600 nm reached to a level of 0.25 to 0.5, and harvested, which was subsequently washed with 0.01M MgCl₂, and centrifuged. To the precipitate thus obtained were added solution containing 0.1M CaCl₂ and 0.05M MgCl₂, and the pHU044 expression vector prepared in Example 4-1, and incubated on ice. The cells were centrifuged again, and dispersed uniformly in the same solution (see: DNA Cloning Vol. I, A Practical Approach, IRL Press, 1985). In this conncetion, all solutions and tubes were used after cooling at 0° C.

And then, 0.2 ml of the cell suspension thus obtained was added to petri dishes coated with liquid LB media containing 12.5 μg/ml of tetracycline, and cultured at 37° C. overnight to obtain transformant of E. coli JM101 harboring pHU044. The transformant thus prepared was designated as Escherichia coli DW/HU-044, and deposited with the Korean Culture Center of Microorganisms (KCCM), an international depositary authority located at College of Eng., Yonsei University, Sodaemun-gu, Seoul, Korea, under an accession No. KCCM-10124 on Mar. 12, 1997.

### EXAMPLE 5-2

### Preparation of a Transformant Containing pHC033

In order to transform host cell with the pHC033 expression vector, E. coli JM101 was first inoculated in liquid LB medium, cultured at 37° C. until absorbance at 600 nm reached to a level of 0.25 to 0.5, and harvested, which was subsequently washed with 0.1M MgCl₂, and centrifuged. To the precipitate thus obtained were added solution containing 0.1M CaCl₂ and 0.05M MgCl₂, and the pHC033 expression vector prepared in Example 4-2, and incubated on ice. The cells were centrifuged again, and dispersed uniformly in the same solution (see: DNA Cloning Vol. I, A Practical Approach, IRL press, 1985). In this conncetion, all solutions and tubes were used after cooling at 0° C.

And then, 0.2 ml of the cell suspension thus obtained was added to petri dishes coated with liquid LB media containing 12.5 μg/ml of tetracycline, and cultured at 37° C. overnight to obtain transformant of E. coli JM101 harboring pHC033. The transformant thus prepared was designated as Escherichia coli DW/HC-033, and deposited with the Korean Culture Center of Microorganisms (KCCM), an international depositary authority located at College of Eng., Yonsei University, Korea, under an accession No. KCCM-10123 on Mar. 12, 1997.

### EXAMPLE 5-3

### Preparation of a Transformant Expressing alpA-fused Gene

Transformant was prepared in an analogous manner as in Example 5-1, with an exception of employing the expression vector containing a fused gene of alpA gene and A2/B subunit gene of Vibro cholerae toxin which was prepared in Example 4-3.

### EXAMPLE 5-4

### Preparation of a Transformant Expressing alpB-fused Gene

Transformant was prepared in an analogous manner as in Example 5-1, with an exception of employing the expression vector containing a fused gene of alpB gene and A2/B subunit gene of Vibro cholerae toxin which was prepared in Example 4-4.

### EXAMPLE 5-5

### Preparation of a Transformant Expressing fliQ-fused Gene

Transformant was prepared in an analogous manner as in Example 5-1, with an exception of employing the expression vector containing a fused gene of fliQ gene and A2/B subunit gene of Vibro cholerae toxin prepared in Example 4-5.

### EXAMPLE 5-6

### Preparation of a Transformant Expressing babA1-fused Gene

Transformant was prepared in an analogous manner as in Example 5-1, with an exception of employing the expression vector containing a fused gene of babA1 gene and A2/B subunit gene of Vibro cholerae toxin prepared in Example 4-6.

### EXAMPLE 5-7

### Preparation of a Transformant Expressing babA2-fused Gene

Transformant was prepared in an analogous manner as in Example 5-1, with an exception of employing the expression vector containing a fused gene of babA2 gene and A2/B subunit gene of Vibro cholerae toxin prepared in Example 4-7.

### EXAMPLE 5-8

### Preparation of a Transformant Expressing ureC-fused Gene

Transformant was prepared in an analogous manner as in Example 5-1, with an exception of employing the expression vector containing a fused gene of ureC gene and A2/B subunit gene of Vibro cholerae toxin prepared in Example 4-8.

### EXAMPLE 5-9

### Preparation of a Transformant Expressing ureD-fused Gene

Transformant was prepared in an analogous manner as in Example 5-1, with an exception of employing the expression vector containing a fused gene of ureD gene and A2/B subunit gene of Vibro cholerae toxin prepared in Example 4-9.

### EXAMPLE 5-10

### Preparation of a Transformant Expressing ureA-fused Gene

Transformant was prepared in an analogous manner as in Example 5-1, with an exception of employing the expression vector containing a fused gene of ureA gene and A2/B subunit gene of Vibro cholerae toxin prepared in Example 4-10.

### EXAMPLE 5-11

### Preparation of a Transformant Expressing sodB-fused Gene

Transformant was prepared in an analogous manner as in Example 5-1, with an exception of employing the expression vector containing a fused gene of sodB gene and A2/B subunit gene of Vibro cholerae toxin prepared in Example 4-11.

### EXAMPLE 5-12

### Preparation of a Transformant Expressing ureI-fused Gene

Transformant was prepared in an analogous manner as in Example 5-1, with an exception of employing the expression vector containing a fused gene of ureI gene and A2/B subunit gene of Vibro cholerae toxin prepared in Example 4-12.

### EXAMPLE 5-13

### Preparation of a Transformant Expressing ureE-fused Gene

Transformant was prepared in an analogous manner as in Example 5-1, with an exception of employing the expression vector containing a fused gene of ureE gene and A2/B subunit gene of Vibro cholerae toxin prepared in Example 4-13.

### EXAMPLE 5-14

### Preparation of a Transformant Expressing ureF-fused Gene

Transformant was prepared in an analogous manner as in Example 5-1, with an exception of employing the expression vector containing a fused gene of ureF gene and A2/B subunit gene of Vibro cholerae toxin prepared in Example 4-14.

### EXAMPLE 5-15

### Preparation of a Transformant Expressing ureG-fused Gene

Transformant was prepared in an analogous manner as in Example 5-1, with an exception of employing the expression vector containing a fused gene of ureG gene and A2/B subunit gene of Vibro cholerae toxin prepared in Example 4-15.

### EXAMPLE 5-16

### Preparation of a Transformant Expressing ureH-fused Gene

Transformant was prepared in an analogous manner as in Example 5-1, with an exception of employing the expression vector containing a fused gene of ureH gene and A2/B subunit gene of Vibro cholerae toxin prepared in Example 4-16.

### EXAMPLE 5-17

### Preparation of a Transformant Expressing flaA-fused Gene

Transformant was prepared in an analogous manner as in Example 5-1, with an exception of employing the expression vector containing a fused gene of flaA gene and A2/B subunit gene of Vibro cholerae toxin prepared in Example 4-17.

### EXAMPLE 5-18

### Preparation of a Transformant Expressing flaB-fused Gene

Transformant was prepared in an analogous manner as in Example 5-1, with an exception of employing the expression vector containing a fused gene of flaB gene and A2/B subunit gene of Vibro cholerae toxin prepared in Example 4-18.

### EXAMPLE 5-19

### Preparation of a Transformant Expressing catA-fused Gene

Transformant was prepared in an analogous manner as in Example 5-1, with an exception of employing the expression vector containing a fused gene of catA gene and A2/B subunit gene of Vibro cholerae toxin prepared in Example 4-19.

### EXAMPLE 5-20

### Preparation of a Transformant Expressing vacA-fused Gene

Transformant was prepared in an analogous manner as in Example 5-1, with an exception of employing the expression vector containing a fused gene of vacA gene and A2/B subunit gene of Vibro cholerae toxin prepared in Example 4-20.

### EXAMPLE 5-21

### Preparation of a Transformant Expressing babB-fused Gene

Transformant was prepared in an analogous manner as in Example 5-1, with an exception of employing the expression vector containing a fused gene of babB gene and A2/B subunit gene of Vibro cholerae toxin prepared in Example 4-21.

### EXAMPLE 6

### Expression of Chimeric Proteins

### EXAMPLE 6-1

Expression of a chimeric protein in transformant E. coli DW/HU-044

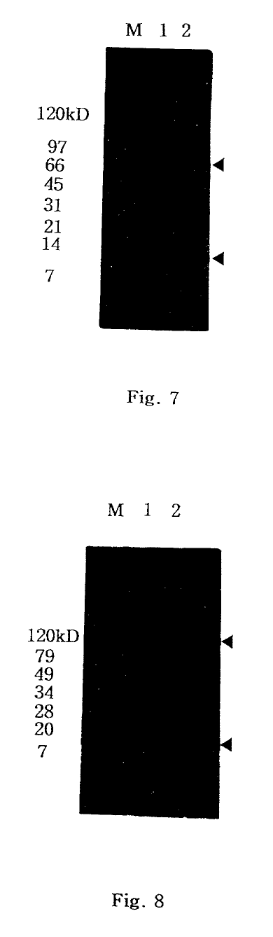

A transformant E. coli DW/HU-044 was inoculated in about 3 ml of a medium which is disclosed in Table 1 below, and overnight cultured at 37° C. and 250 rpm, and 0.5 ml of the culture was inoculated in about 50 ml of the same medium and cultured at 37° C. while shaking at 250 rpm to reach 1.8 to 2.0 of the absorbance at 600 nm. Then, to the culture, was added 0.25 ml of IPTG (isopropyl β-D-thiogalactoside) and cultured at 37° C. at 250 rpm for 24 hours to induce recombinant protein, centrifuged to collect cells, suspended in a buffer solution (10 mM Tris-HCl (pH 8.0) containing 0.1% Triton X-100, 2 mM EDTA and 1 mM PMSF) to lyse cells, and electrophoresed on 15% SDS-PAGE (see: FIG. 7).

TABLE 1
Composition of medium for transformant culture

| Composition of medium | ˜50 ml of medium for | ˜3 ml of medium |
|---|---|---|
| (per liter) | expression | for expression |
| Main medium | | |
| yeast extract 20 g | 44 ml | 2.7 ml |
| casamino acid l0 g | | |
| MgSO₄.7H₂O 0.224 g | | |
| CaCl₂.2 H₂O 0.01 g | | |
| 10 X phosphate buffer | | |
| (100 ml) | | |
| KH₂PO₄ 3 g | 5 ml | 0.3 ml |
| Na₂HPO₄ 4 q | | |
| (NH₄)₂HPO₄ 2.5 g | | |
| 25% Glucose | 1 ml | 0.06 ml |
| Tetracycline | 0.l ml | 0.006 ml |
| (12.5 μg/ml) | | |

In FIG. 7, lane M shows molecular size-marker, lane 1 shows cell lysate before IPTG induction; lane 2 shows cell lysate of 24 hrs cultured cells after IPTG induction; top arrow indicates locus of a chimeric protein containing ureB of H. pylori and A2 subunit of Vibro cholerae toxin; and, bottom arrow indicates locus of B subunit of Vibro cholerae toxin.

As shown in FIG. 7, it was found that the transformed E. coli DW/HU-044 successfully expresses a chimeric protein, which is designated as ‘UreB/CTXA2B’.

### EXAMPLE 6-2

### Expression of a Chimeric Protein in Transformant E. coli DW/HC-033

A transformant E. coli DW/HC-033 was cultured similarly as in Example 6-1, and harvested after cetrifugation, suspended in a buffer solution (10 mM Tris-HCl (pH 8.0) containing 0.1% Triton X-100, 2 mM EDTA and 1 mM PMSF) to lyse cells, and electrophoresed on 15% SDS-PAGE (see: FIG. 8).

In FIG. 8, lane M shows molecular size marker, lane 1 shows cell lysate before IPTG induction; lane 2 shows cell lysate of 24 hrs cultured cells after IPTG induction; top arrow indicates locus of a chimeric protein containing cagA of H. pylori and A2 subunit of Vibro cholerae toxin; and, bottom arrow indicates locus of B subunit of Vibro cholerae toxin.

As shown in FIG. 8, it was found that the transformed E. coli DW/HC-033 successfully expresses a chimeric protein, which is designated as ‘CagA/CTXA2B’.

### EXAMPLE 6-3

### Expression of AlpA/CTXA2B in Transformant

The transformant E. coli prepared in Example 5-3 was cultured in an analogous manner as in Example 6-1, to express desired recombinant protein, which is designated as ‘AlpA/CTXA2B’.

### EXAMPLE 6-4

### Expression of AlpB/CTXA2B in Transformant

The transformant E. coli prepared in Example 5-4 was cultured in an analogous manner as in Example 6-1, to express desired recombinant protein, which is designated as ‘AlpB/CTXA2B’.

### EXAMPLE 6-5

### Expression of FliQ/CTXA2B in Transformant

The transformant E. coli prepared in Example 5-5 was cultured in an analogous manner as in Example 6-1, to express desired recombinant protein, which is designated as ‘FliQ/CTXA2B’.

### EXAMPLE 6-6

### Expression of BabA1/CTXA2B in Transformant

The transformant E. coli prepared in Example -5-6 was cultured in an analogous manner as in Example 6-1, to express desired recombinant protein, which is designated as ‘BabA1/CTXA2B’.

### EXAMPLE 6-7

### Expression of BabA2/CTXA2B in Transformant

The transformant E. coli prepared in Example 5-7 was cultured in an analogous manner as in Example 6-1, to express desired recombinant protein, which is designated as ‘BabA2/CTXA2B’.

### EXAMPLE 6-8

### Expression of UreC/CTXA2B in Transformant

The transformant E. coli prepared in Example 5-8 was cultured in an analogous manner as in Example 6-1, to express desired recombinant protein, which is designated as ‘UreC/CTXA2B’.

### EXAMPLE 6-9

### Expression of in UreD/CTXA2B Transformant

The transformant E. coli prepared in Example 5-9 was cultured in an analogous manner as in Example 6-1, to express desired recombinant protein, which is designated as ‘UreD/CTXA2B’.

### EXAMPLE 6-10

### Expression of UreA/CTXA2B in Transformant

The transformant E. coli prepared in Example 5-10 was cultured in an analogous manner as in Example 6-1, to express desired recombinant protein, which is designated as ‘UreA/CTXA2B’.

### EXAMPLE 6-11

### Expression of SodB/CTXA2B in Transformant

The transformant E. coli prepared in Example 5-11 was cultured in an analogous manner as in Example 6-1, to express desired recombinant protein, which is designated as ‘SodB/CTXA2B’.

### EXAMPLE 6-12

### Expression of UreI/CTXA2B in Transformant

The transformant E. coli prepared in Example 5-12 was cultured in an analogous manner as in Example 6-1, to express desired recombinant protein, which is designated as ‘UreI/CTXA2B’.

### EXAMPLE 6-13

### Expression of UreE/CTXA2B in Transformant

The transformant E. coli prepared in Example 5-13 was cultured in an analogous manner as in Example 6-1, to express desired recombinant protein, which is designated as ‘UreE/CTXA2B’.

### EXAMPLE 6-14

### Expression of UreF/CTXA2B in Transformant

The transformant E. coli prepared in Example 5-14 was cultured in an analogous manner as in Example 6-1, to express desired recombinant protein, which is designated as ‘UreF/CTXA2B’.

### EXAMPLE 6-15

### Expression of UreG/CTXA2B in Transformant

The transformant E. coli prepared in Example 5-15 was cultured in an analogous manner as in Example 6-1, to express desired recombinant protein, which is designated as ‘UreG/CTXA2B’.

### EXAMPLE 6-16

### Expression of UreH/CTXA2B in Transformant

The transformant E. coli prepared in Example 5-16 was cultured in an analogous manner as in Example 6-1, to express desired recombinant protein, which is designated as ‘UreH/CTXA2B’.

### EXAMPLE 6-17

### Expression of FlaA/CTXA2B in Transformant

The transformant E. coli prepared in Example 5-17 was cultured in an analogous manner as in Example 6-1, to express desired recombinant protein, which is designated as ‘FlaA/CTXA2B’.

### EXAMPLE 6-18

### Expression of FlaB/CTXA2B in Transformant

The transformant E. coli prepared in Example 5-18 was cultured in an analogous manner as in Example 6-1, to express desired recombinant protein, which is designated as ‘FlaB/CTXA2B’.

### EXAMPLE 6-19

### Expression of CatA/CTXA2B in Transformant

The transformant E. coli prepared in Example 5-19 was cultured in an analogous manner as in Example 6-1, to express desired recombinant protein, which is designated as ‘CatA/CTXA2B’.

### EXAMPLE 6-20

### Expression of VacA/CTXA2B in Transformant

The transformant E. coli prepared in Example 5-20 was cultured in an analogous manner as in Example 6-1, to express desired recombinant protein, which is designated as ‘VacA/CTXA2B’.

### EXAMPLE 6-21

### Expression of BabB/CTXA2B in Transformant

The transformant E. coli prepared in Example 5-21 was cultured in an analogous manner as in Example 6-1, to express desired recombinant protein, which is designated as ‘BabB/CTXA2B’.

### EXAMPLE 7

### Purification of Chimeric Proteins from the Culture

### EXAMPLE 7-1

### Purification of UreB/CTXA2B Chimeric Protein

The E. coli DW/HU-044(KCCM-10124) was cultured in a LB medium and further cultured for 4 hours after IPTG induction.

The cultured cells were harvested by centrifugation and lysed with lysozyme. The lysed cells were washed several times with 0.5% Triton X-100, and washed with 8M urea to remove contaminated proteins. Then, inclusion bodies were dissolved in 8M urea and 0.1M DTT, diluted with glutathione redox buffer to refold the UreB/CTXA2B protein. Centrifugation was carried out to obtain the refolded chimeric protein, and size-exclusion chromatography was performed to obtain the UreB/CTXA2B chimeric protein only. SDS-PAGE, Western-blot and G_{M1}-ganglioside analysis confirmed that the obtained protein is UreB/CTXA2B chimeric protein.

### EXAMPLE 7-2

### Purification of CagA/CTXA2B Chimeric Protein

The E. coli DW/HC-033 (KCCM-10123) was cultured in a LB medium and further cultured for 4 hours after IPTG induction. The cultured cells were harvested by centrifugation and lysed with lysozyme. The lysed cells were washed several times with 0.5% Triton X-100, and washed with 8M urea to remove contaminated proteins. Then, inclusion bodies were dissolved in 8M urea and 0.1M DTT, diluted with glutathione redox buffer to refold the CagA/CTXA2B protein. Centrifugation was carried out to obtain the refolded chimeric protein, and size-exclusion chromatography was performed to obtain the CagA/CTXA2B chimeric protein only. SDS-PAGE, Western-blot and G_{M1}-ganglioside analysis confirmed that the obtained protein is CagA/CTXA2B chimeric protein.

### EXAMPLE 7-3

### Purification of AlpA/CTXA2B Chimeric Protein

Chimeric protein was expressed in Example 6-3, which was prepared and identified as AlpA/CTXA2B in accordance with the method described in Example 7-1.

### EXAMPLE 7-4

### Purification of AlpB/CTXA2B Chimeric Protein

Chimeric protein was expressed in Example 6-4, which was prepared and identified as AlpB/CTXA2B in accordance with the method described in Example 7-1.

### EXAMPLE 7-5

### Purification of FliQ/CTXA2B Chimeric Protein

Chimeric protein was expressed in Example 6-5, which was prepared and identified as FliQ/CTXA2B in accordance with the method described in Example 7-1.

### EXAMPLE 7-6

### Purification of BabA1/CTXA2B Chimeric Protein

Chimeric protein was expressed in Example 6-6, which was prepared and identified as BAbA1/CTXA2B in accordance with the method described in Example 7-1.

### EXAMPLE 7-7

### Purification of BabA2/CTXA2B Chimeric Protein

Chimeric protein was expressed in Example 6-7, which was prepared and identified as BabA2/CTXA2B in accordance with the method described in Example 7-1.

### EXAMPLE 7-8

### Purification of UreC/CTXA2B Chimeric Protein

Chimeric protein was expressed in Example 6-8, which was prepared and identified as UreC/CTXA2B in accordance with the method described in Example 7-1.

### EXAMPLE 7-9

### Purification of UreD/CTXA2B Chimeric Protein

Chimeric protein was expressed in Example 6-9, which was prepared and identified as UreD/CTXA2B in accordance with the method described in Example 7-1.

### EXAMPLE 7-10

### Purification of UreA/CTXA2B Chimeric Protein

Chimeric protein was expressed in Example 6-10, which was prepared and identified as UreA/CTXA2B in accordance with the method described in Example 7-1.

### EXAMPLE 7-11

### Purification of SodB/CTXA2B Chimeric Protein

Chimeric protein was expressed in Example 6-11, which was prepared and identified as SodB/CTXA2B in accordance with the method described in Example 7-1.

### EXAMPLE 7-12

### Purification of UreI/CTXA2D Chimeric Protein

Chimeric protein was expressed in Example 6-12, which was prepared and identified as UreI/CTXA2B in accordance with the method described in Example 7-1.

### EXAMPLE 7-13

### Purification of UreE/CTXA2B Chimeric Protein

Chimeric protein was expressed in Example 6-13, which was prepared and identified as UreE/CTXA2B in accordance with the method described in Example 7-1.

### EXAMPLE 7-14

### Purification of UreF/CTXA2B Chimeric Protein

Chimeric protein was expressed in Example 6-14, which was prepared and identified as UreF/CTXA2B in accordance with the method described in Example 7-1.

### EXAMPLE 7-15

### Purification of UreG/CTXA2B Chimeric Protein

Chimeric protein was expressed in Example 6-15, which was prepared and identified as UreG/CTXA2B in accordance with the method described in Example 7-1.

### EXAMPLE 7-16

### Purification of UreH/CTXA2B Chimeric Protein

Chimeric protein was expressed in Example 6-16, which was prepared and identified as UreH/CTXA2B in accordance with the method described in Example 7-1.

### EXAMPLE 7-17

### Purification of FlaA/CTXA2B Chimeric Protein

Chimeric protein was expressed in Example 6-17, which was prepared and identified as FlaA/CTXA2B in accordance with the method described in Example 7-1.

### EXAMPLE 7-18

### Purification of FlaB/CTXA2B Chimeric Protein

Chimeric protein was expressed in Example 6-18, which was prepared and identified as FlaB/CTXA2B in accordance with the method described in Example 7-1.

### EXAMPLE 7-19

### Purification of CatA/CTXA2B Chimeric Protein

Chimeric protein was expressed in Example 6-19, which was prepared and identified as CatA/CTXA2B in accordance with the method described in Example 7-1.

### EXAMPLE 7-20

### Purification of VacA/CTXA2B Chimeric Protein

Chimeric protein was expressed in Example 6-20, which was prepared and identified as VacA/CTXA2B in accordance with the method described in Example 7-1.

### EXAMPLE 7-21

### Purification of BabB/CTXA2B Chimeric Protein

Chimeric protein was expressed in Example 6-21, which was prepared and identified as BabB/CTXA2B in accordance with the method described in Example 7-1.

### EXAMPLE 8

### Immunological Reaction of the Chimeric Proteins

### EXAMPLE 8-1

### Immunological Reaction of the Chimeric Protein (UreB/CTXA2B)

In order to determine an antibody production rate of the UreB/CTXA2B chimeric protein obtained in Example 7-1, an animal experiment was carried out, in accordance with a protocol of the National Institutes of Health (NIH): That is, taking 4 Balb/C mice of 11 to 12-week as one experimental group, 100 μg of the UreB/CTXA2B chimeric protein dissolved in 0.5 ml of 350 mM NaHCO₃, 100 μg of UreB dissolved in 0.5 ml of 350 mM NaHCO₃, and only 0.5 ml of 350 mM NaHCO₃ as a control were administered orally into stomach three times at 10-day intervals for immunization, respectively. The test animals were starved for 2 hours before the oral administration and for 1 hour after the oral administration. Sera were obtained by tail bleeding at 1 day before immunization (0-day) and every week after immunization (8, 18, 28-day). Antibodies of extract of gastric juice were prepared by administering 0.5 ml of a lavage solution (containing of 25 mM NaCl, 40 mM Na₂SO₄, 10 mM KCl, 20 mM NaHCO₃ and 48.5 mM polyethyleneglycol) four times at 15-minute intervals into mice, injecting 0.2 ml of pilocarpine (0.5 mg/ml) peritoneally at 30 minutes after the last administration and obtaining extracts of gastric juice from mice at 30 minutes after injection.

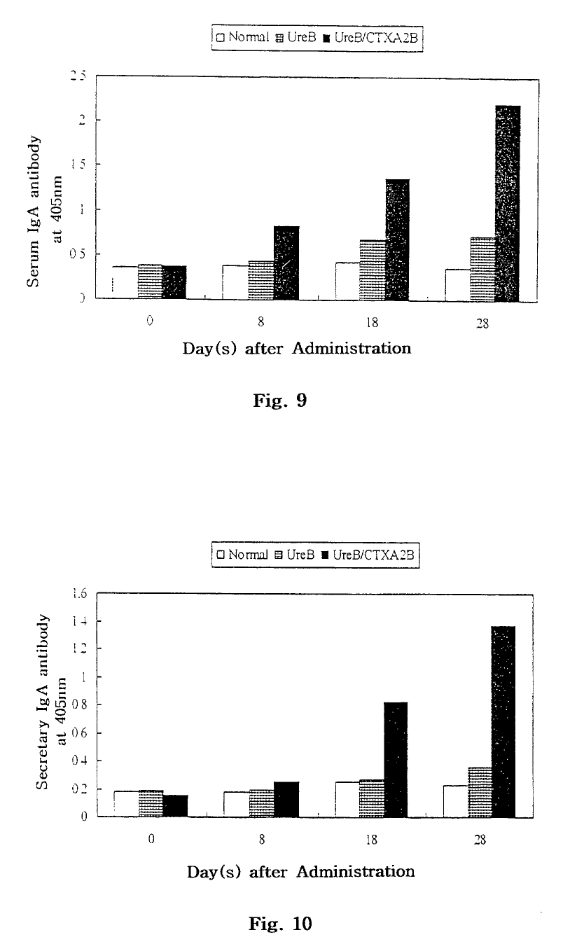

Quantitation of the antibody produced by UreB/CTXA2B was carried out using ELISA as followings: That is, after sera and extract of gastric juice were treated into a 96-well plate treated with goat anti-mouse IgG and IgA antibodies, goat peroxidase-conjugated antibodies against each isotype of mouse antibody as secondary antibodies were treated. Absorbance at 405 nm was measured using p-nitrophenyl phosphate as substrates of peroxidase to determine an antibody production rate. As a result, it was found that: when the UreB/CTXA2B chimeric protein was administered, the amount of serum IgG increased remarkably after 18 days and increased 3-fold or more compared with mice administered with only UreB (see: FIG. 9). Also, it was revealed that amount of IgA in extract of gastric juice increased 3-fold or more compared with mice administered with only UreB (see: FIG. 10).

### EXAMPLE 8-2

### Immunological Reaction of the Chimeric Protein (CatA/CTXA2B)

In order to determine an antibody production rate of the CatA/CTXA2B chimeric protein obtained in Example 7-2, an animal experiment was carried out, in accordance with a protocol of the National Institutes of Health (NIH): That is, taking 4 Balb/C mice of 11 to 12-week as one experimental group, 100 μg of the CatA/CTXA2B chimeric protein dissolved in 0.5 ml of 350 mM NaHCO₃, 100 μg of CatA dissolved in 0.5 ml of 350 mM NaHCO₃, and only 0.5 ml of 350 mM NaHCO₃ as a control were administered orally into stomach three times at 10-day intervals for immunization, respectively. The test animals were starved for 2 hours before the oral administration and for 1 hour after the oral administration. Sera were obtained by tail bleeding at 1 day before immunization (0-day) and every week after immunization (8, 18, 28-day). Antibodies of extract of gastric juice were prepared by administering 0.5 ml of a lavage solution (containing of 25 mM NaCl, 40 mM Na₂SO₄, 10 mM KCl, 20 mM NaHCO₃ and 48.5 mM polyethyleneglycol) four times at 15-minute intervals into mice, injecting 0.2 ml of pilocarpine (0.5 mg/ml) peritoneally at 30 minutes after the last administration and obtaining extracts of gastric juice from mice at 30 minutes after injection.

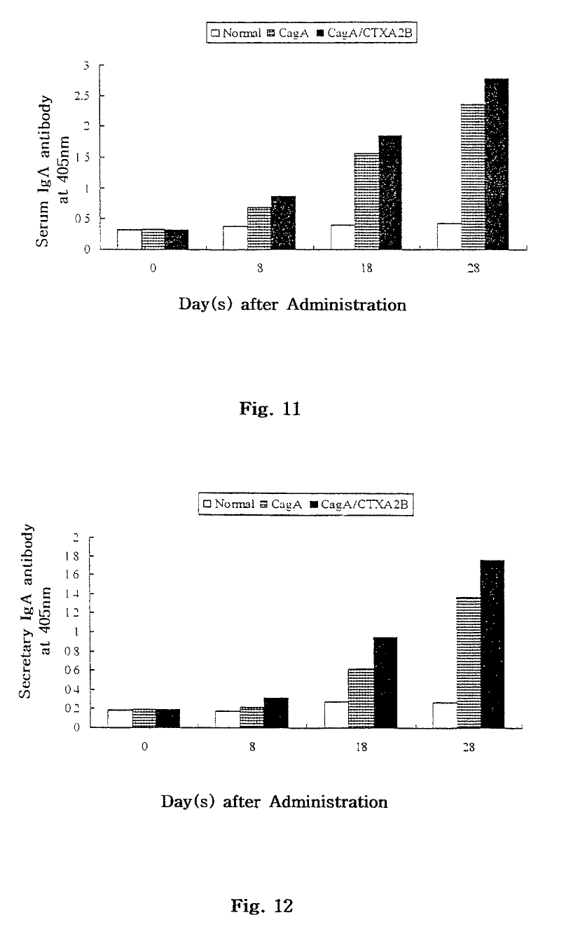

Quantitation of the antibody produced by CatA/CTXA2B was carried out using ELISA as followings: That is, after sera and extract of gastric juice were treated into a 96-well plate treated with goat anti-mouse IgG and IgA antibodies, goat peroxidase-conjugated antibodies against each isotype of mouse antibody as secondary antibodies were treated. Absorbance at 405 nm was measured using p-nitrophenyl phosphate as substrates of peroxidase to determine an antibody production rate. As a result, it was found that: when the CatA/CTXA2B chimeric protein was administered, the amount of serum IgG increased remarkably after 18 days and increased 0.3-fold or more compared with mice administered with CatA only (see: FIG. 11). Also, it was revealed that amount of IgA in extract of gastric juice increased 0.3-fold or more compared with mice administered with only CatA (see: FIG. 12).

### EXAMPLE 8-3

### Immunological Reaction of the Chimeric Protein (AlpA/CTXA2B)

The animal experiment and antibody quantitation were carried out in an analogous manner as in Example 8-1, with an exception that AlpA/CTXA2B chimeric protein was employed for the determination of antibody productivity of AlpA/CTXA2B prepared in Example 7-3. As a result, it was found that: when the AlpA/CTXA2B chimeric protein was administered, the amount of serum IgG increased remarkably after 18 days compared with mice administered with only AlpA. Also, it was revealed that amount of IgA in extract of gastric juice increased compared with mice administered with only AlpA.

### EXAMPLE 8-4

### Immunological Reaction of the Chimeric Protein (AlpB/CTXA2B)

The animal experiment and antibody quantitation were carried out in an analogous manner as in Example 8-1, with an exception that AlpB/CTXA2B chimeric protein was employed for the determination of antibody productivity of AlpB/CTXA2B prepared in Example 7-4. As a result, it was found that: when the AlpB/CTXA2B chimeric protein was administered, the amount of serum IgG increased remarkably after 18 days compared with mice administered with only AlpB. Also, it was revealed that amount of IgA in extract of gastric juice increased compared with mice administered with only AlpB.

### EXAMPLE 8-5

### Immunological Reaction of the Chimeric Protein (FliQ/CTXA2B)

The animal experiment and antibody quantitation were carried out in an analogous manner as in Example 8-1, with an exception that FliQ/CTXA2B chimeric protein was employed for the determination of antibody productivity of FliQ/CTXA2B prepared in Example 7-5. As a result, it was found that: when the FliQ/CTXA2B chimeric protein was administered, the amount of serum IgG increased remarkably after 18 days compared with mice administered with only AlpA. Also, it was revealed that amount of IgA in extract of gastric juice increased compared with mice administered with only FliQ.

### EXAMPLE 8-6

### Immunological Reaction of the Chimeric Protein (BabA1/CTXA2B)

The animal experiment and antibody quantitation were carried out in an analogous manner as in Example 8-1, with an exception that BabA1/CTXA2B chimeric protein was employed for the determination of antibody productivity of BabA1/CTXA2B prepared in Example 7-6. As a result, it was found that: when the BabA1/CTXA2B chimeric protein was administered, the amount of serum IgG increased remarkably after 18 days compared with mice administered with only BabA1. Also, it was revealed that amount of IgA in extract of gastric juice increased compared with mice administered with only BabA1.

### EXAMPLE 8-7

### Immunological Reaction of the Chimeric Protein (BabA2/CTXA2B)

The animal experiment and antibody quantitation were carried out in an analogous manner as in Example 8-1, with an exception that BabA2/CTXA2B chimeric protein was employed for the determination of antibody productivity of BabA2/CTXA2B prepared in Example 7-7. As a result, it was found that: when the BabA2/CTXA2B chimeric protein was administered, the amount of serum IgG increased remarkably after 18 days compared with mice administered with only BabA2. Also, it was revealed that amount of IgA in extract of gastric juice increased compared with mice administered with only BabA2.

### EXAMPLE 8-8

### Immunological Reaction of the Chimeric Protein (UreC/CTXA2B)

The animal experiment and antibody quantitation were carried out in an analogous manner as in Example 8-1, with an exception that UreC/CTXA2B chimeric protein was employed for the determination of antibody productivity of UreC/CTXA2B prepared in Example 7-8. As a result, it was found that: when the UreC/CTXA2B chimeric protein was administered, the amount of serum IgG increased remarkably after 18 days compared with mice administered with only UreC. Also, it was revealed that amount of IgA in extract of gastric juice increased compared with mice administered with only UreC.

### EXAMPLE 8-9

### Immunological Reaction of the Chimeric Protein (UreD/CTXA2B)

The animal experiment and antibody quantitation were carried out in an analogous manner as in Example 8-1, with an exception that UreD/CTXA2B chimeric protein was employed for the determination of antibody productivity of UreD/CTXA2B prepared in Example 7-9. As a result, it was found that: when the UreD/CTXA2B chimeric protein was administered, the amount of serum IgG increased remarkably after 18 days compared with mice administered with only UreD. Also, it was revealed that amount of IgA in extract of gastric juice increased compared with mice administered with only UreD.

### EXAMPLE 8-10

### Immunological Reaction of the Chimeric Protein (UreA/CTXA2B)

The animal experiment and antibody quantitation were carried out in an analogous manner as in Example 8-1, with an exception that UreA/CTXA2B chimeric protein was employed for the determination of antibody productivity of UreA/CTXA2B prepared in Example 7-10. As a result, it was found that: when the UreA/CTXA2B chimeric protein was administered, the amount of serum IgG increased remarkably after 18 days compared with mice administered with only UreA. Also, it was revealed that amount of IgA in extract of gastric juice increased compared with mice administered with only UreA.

### EXAMPLE 8-11

### Immunological Reaction of the Chimeric Protein (SodB/CTXA2B)

The animal experiment and antibody quantitation were carried out in an analogous manner as in Example 8-1, with an exception that SodB/CTXA2B chimeric protein was employed for the determination of antibody productivity of SodB/CTXA2B prepared in Example 7-11. As a result, it was found that: when the SodB/CTXA2B chimeric protein was administered, the amount of serum IgG increased remarkably after 18 days compared with mice administered with only SodB. Also, it was revealed that amount of IgA in extract of gastric juice increased compared with mice administered with only SodB.

### EXAMPLE 8-12

### Immunological Reaction of the Chimeric Protein (UreI/CTXA2B)

The animal experiment and antibody quantitation were carried out in an analogous manner as in Example 8-1, with an exception that UreI/CTXA2B chimeric protein was employed for the determination of antibody productivity of UreI/CTXA2B prepared in Example 7-12. As a result, it was found that: when the UreI/CTXA2B chimeric protein was administered, the amount of serum IgG increased remarkably after 18 days compared with mice administered with only UreI. Also, it was revealed that amount of IgA in extract of gastric juice increased compared with mice administered with only UreI.

### EXAMPLE 8-13

### Immunological Reaction of the Chimeric Protein (UreE/CTXA2B)

The animal experiment and antibody quantitation were carried out in an analogous manner as in Example 8-1, with an exception that UreE/CTXA2B chimeric protein was employed for the determination of antibody productivity of UreE/CTXA2B prepared in Example 7-13. As a result, it was found that: when the UreE/CTXA2B chimeric protein was administered, the amount of serum IgG increased remarkably after 18 days compared with mice administered with only UreE. Also, it was revealed that amount of IgA in extract of gastric juice increased compared with mice administered with only UreE.

### EXAMPLE 8-15

### Immunological Reaction of the Chimeric Protein (UreG/CTXA2B)

The animal experiment and antibody quantitation were carried out in an analogous manner as in Example 8-1, with an exception that UreG/CTXA2B chimeric protein was employed for the determination of antibody productivity of UreG/CTXA2B prepared in Example 7-15. As a result, it was found that: when the UreG/CTXA2B chimeric protein was administered, the amount of serum IgG increased remarkably after 18 days compared with mice administered with only UreG. Also, it was revealed that amount of IgA in extract of gastric juice increased compared with mice administered with only UreG.

### EXAMPLE 8-16

### Immunological Reaction of the Chimeric Protein (UreH/CTXA2B)

The animal experiment and antibody quantitation were carried out in an analogous manner as in Example 8-1, with an exception that UreH/CTXA2B chimeric protein was employed for the determination of antibody productivity of UreH/CTXA2B prepared in Example 7-16. As a result, it was found that: when the UreH/CTXA2B chimeric protein was administered, the amount of serum IgG increased remarkably after 18 days compared with mice administered with only UreH. Also, it was revealed that amount of IgA in extract of gastric juice increased compared with mice administered with only UreH.

### EXAMPLE 8-17

### Immunological Reaction of the Chimeric Protein (FlaA/CTXA2B)

The animal experiment and antibody quantitation were carried out in an analogous manner as in Example 8-1, with an exception that FlaA/CTXA2B chimeric protein was employed for the determination of antibody productivity of FlaA/CTXA2B prepared in Example 7-17. As a result, it was found that: when the FlaA/CTXA2B chimeric protein was administered, the amount of serum IgG increased remarkably after 18 days compared with mice administered with only FlaA. Also, it was revealed that amount of IgA in extract of gastric juice increased compared with mice administered with only FlaA.

### EXAMPLE 8-18

### Immunological Reaction of the Chimeric Protein (FlaB/CTXA2B)

The animal experiment and antibody quantitation were carried out in an analogous manner as in Example 8-1, with an exception that FlaB/CTXA2B chimeric protein was employed for the determination of antibody productivity of FlaB/CTXA2B prepared in Example 7-18. As a result, it was found that: when the FlaB/CTXA2B chimeric protein was administered, the amount of serum IgG increased remarkably after 18 days compared with mice administered with only FlaB. Also, it was revealed that amount of IgA in extract of gastric juice increased compared with mice administered with only FlaB.

### EXAMPLE 8-19

### Immunological Reaction of the Chimeric Protein (CatA/CTXA2B)

The animal experiment and antibody quantitation were carried out in an analogous manner as in Example 8-1, with an exception that CatA/CTXA2B chimeric protein was employed for the determination of antibody productivity of CatA/CTXA2B prepared in Example 7-19. As a result, it was found that: when the CatA/CTXA2B chimeric protein was administered, the amount of serum IgG increased remarkably after 18 days compared with mice administered with only CatA. Also, it was revealed that amount of IgA in extract of gastric juice increased compared with mice administered with only CatA.

### EXAMPLE 8-20

### Immunological Reaction of the Chimeric Protein (VacA/CTXA2B)

The animal experiment and antibody quantitation were carried out in an analogous manner as in Example 8-1, with an exception that VacA/CTXA2B chimeric protein was employed for the determination of antibody productivity of VacA/CTXA2B prepared in Example 7-20. As a result, it was found that: when the VacA/CTXA2B chimeric protein was administered, the amount of serum IgG increased remarkably after 18 days compared with mice administered with only VacA. Also, it was revealed that amount of IgA in extract of gastric juice increased compared with mice administered with only VacA.

### EXAMPLE 8-21

### Immunological Reaction of the Chimeric Protein (BabB/CTXA2B)

The animal experiment and antibody quantitation were carried out in an analogous manner as in Example 8-1, with an exception that BabB/CTXA2B chimeric protein was employed for the determination of antibody productivity of BabB/CTXA2B prepared in Example 7-8. As a result, it was found that: when the BabB/CTXA2B chimeric protein was administered, the amount of serum IgG increased remarkably after 18 days compared with mice administered with only BabB. Also, it was revealed that amount of IgA in extract of gastric juice increased compared with mice administered with only BabB.

### EXAMPLE 9

### Effect of the Chimeric Proteins as a Vaccine for H. pylori-Associated Disease

### EXAMPLE 9-1

### Effect of the UreB/CTXA2B Chimeric Protein as a Vaccine

Effect of the UreB/CTXA2B chimeric protein as an active ingredient for a potential vaccine against H. pylori was determined by investigating infection of mice immunized with the UreB/CTXA2B chimeric protein and UreB, respectively, with H. pylori.

After 11 to 12 C57BL/6 mice were taken as one experimental group, 100 μg of the UreB/CTXA2B chimeric protein dissolved in 0.5 ml of a physiological saline, 100 μg of UreB dissolved in 0.5 ml of a physiological saline, and only 0.5 ml of a physiological saline as a control were administered orally into stomach three times at 1-week intervals using polyethylene catecher, respectively. At 7 days after the last administration, H. pylori Q-35(obtainable from the College of Medicine, Kyungsang National University, Korea) strain was suspended in 0.1 ml of a physiological saline in a concentration of 10⁷CFU and administered into mice three times at 2-day intervals using polyethylene catecher.

After 2 weeks, pylori of stomachs of all mice were cut in a size of 0.5 cm×0.5 cm and soaked in 1 ml of a sterilized Brain Heart Infusion broth (Difco, U.S.A.). After each sample was diluted with a sterilized physiological saline in a serial dilution of 10-fold, 100 μl of the sample was inoculated in a medium(Blood Agar Base No.2 containing 5% horse serum, 10 mg/ml vancomycin, 5 mg/ml trimethoprim and 4 mg/ml amphotericin B) and cultured at 37° C. for 5 days in a CO₂ incubator (10% CO₂, humidity of 90% or more). After cultivation, number of colonies showing appearance of H. pylori was measured and the corresponding colonies were transferred onto a fresh medium and cultured for 3 days.

The cultured strains were suspended in 500 ml of a physiological saline and catalase, oxidase and urease reactions were carried out as followings: First, 10 μl of each sample was added to 1 ml of an urease-detecting reagent (20 g/l urea, 0.05% (w/v) phenolred, 0.044 g/l NaH₂PO₄H₂O, 1.02 g/l Na₂HPO₄, 0.2 g/l NaN₃), vortexed well, and incubated at room temperature for 4 hours, and its absorbance at 550 nm was measured. In this connection, a sample having a value of 0.1 or more higher than a control without a sample was considered as a sample showing a positive reaction. On the other hand, in order to detect catalase, one drop of a sample was added onto a slide glass and one drop of 3% H₂O₂ was dropped onto it. In this connection, a reaction showing generation of gas and bubbles was considered as a positive reaction. Also, in order to detect oxidase, one drop of a sample was added onto a filter paper and one drop of 1% N,N′-tetramethyl-p-phenylenediamine dissolved in isoamylalcohol was dropped onto it. In this connection, a reaction showing a purple color within several minutes was considered as a positive reaction.

A sample showing positive reactions in all three experiments mentioned as above was regarded as a sample infected with H. pylori. As can be seen in Table 2 below, the experiments revealed that the experimenatl groups administered with UreB/CTXA2B and UreB showed prevention rate of 75% and 27%, respectively. On the other hand, it was found that all mice of the control group administered with only a physiological saline were infected with H. pylori, which showed no preventive effect.

TABLE 2
Infection of mice immunized with the UreB/CTXA2B
chimeric protein and UreB, respectively,
with H. pylori

| | | Number of mice | |
|---|---|---|---|
| | | showing | |
| | | formation of | |
| Experimental | Infecting | colonies/number | Prevention |
| group | strain | of all mice | rate (%) |
| Control | Q-35 | 12/12 | 0 |
| UreB | Q-35 | 8/11 | 27 |
| UreB/CTXA2B | Q-35 | 3/12 | 75 |
| chimeric | | | |
| protein | | | |

### EXAMPLE 9-2

### Effect of the CagA/CTXA2B Chimeric Protein as a Vaccine

Effect of the CagA/CTXA2B chimeric protein as an active ingredient for a potential vaccine against H. pylori was determined by investigating infection of mice immunized with the CagA/CTXA2B chimeric protein and CagA, respectively, with H. pylori.

After 8 to 11 C57BL/6 mice were taken as one experimental group, 100 ug of the CagA/CTXA2B chimeric protein dissolved in 0.5 ml of a physiological saline, 100 ug of CagA dissolved in 0.5 ml of a physiological saline, and only 0.5 ml of a physiological saline as a control were administered orally into stomach three times at 1-week intervals using polyethylene catecher, respectively. At 7 days after the last administration, H. pylori(Q-35, ATCC 11637) strain was suspended in 0.1 ml of a physiological saline in a concentration of 10⁷CFU and administered into mice three times at 2-day intervals using polyethylene catecher.

After 2 weeks, H. pylori of stomachs of all mice were cut in a size of 0.5 cm×0.5 cm and soaked in 1 ml of a sterilized Brain Heart Infusion broth (Difco, U.S.A.). After each sample was diluted with a sterilized physiological saline in a serial dilution of 10-fold, 100 μl of the sample was inoculated in a medium (Blood Agar Base No.2 containing 5% horse serum, 10 mg/ml vancomycin, 5 mg/ml trimethoprim and 4 mg/ml amphotericin B) and cultured at 37° C. for 5 days in a CO₂ incubator (10% CO₂, humidity of 90% or more). After cultivation, number of colonies showing appearance of H. pylori was measured and the corresponding colonies were transferred onto a fresh medium and cultured for 3 days.

The cultured strains were suspended in 500 ml of a physiological saline and catalase, oxidase and urease reactions were carried out as followings: First, 100 ul of each sample was added to 1 ml of an urease-detecting reagent (20 g/l urea, 0.05% (w/v) phenolred, 0.044 g/l NaH₂PO₄H₂O, 1.02 g/l Na₂HPO₄, 0.2 g/l NaN₃), vortexed well, and incubated at room temperature for 4 hours, and its absorbance at 550 nm was measured. In this connection, a sample having a value of 0.1 or more higher than a control without a sample was considered as a sample showing a positive reaction. On the other hand, in order to detect catalase, one drop of a sample was added onto a slide glass and one drop of 3% H₂O₂ was dropped onto it. In this connection, a reaction showing generation of gas and bubbles was considered as a positive reaction.

Also, in order to detect oxidase, one drop of a sample was added onto a filter paper and one drop of 1% N,N′-tetramethyl-p-phenylenediamine dissolved in isoamylalcohol was dropped onto it. In this connection, a reaction showing a purple color within several minutes was considered as a positive reaction.

A sample showing positive reactions in all three experiments mentioned as above was regarded as a sample infected with H. pylori. As can be seen in Table 4 below, the experiments revealed that the experimenatl groups administered with CagA/CTXA2B and CagA showed prevention rate of 80% and 55%, respectively. On the other hand, all mice of the control group administered with only a physiological saline were infected with H. pylori, which showed no preventive effect.

TABLE 3
Infection of mice immunized with the CagA/CTXA2B
chimeric protein and CagA, respectively,
with H. pylori

| | | Number of mice | |
|---|---|---|---|
| | | showing | |
| | | formation of | |
| Experimental | Infecting | colonies/number | Prevention |
| group | strain | of all mice | rate (%) |
| Control | Q-35 | 8/8 | 0 |
| CagA | Q-35 | 5/11 | 55 |
| CagA/CTXA2B | Q-35 | 2/10 | 80 |
| chimeric | | | |
| prorein | | | |

### EXAMPLE 9-3

### Effect of the AlpA/CTXA2B Chimeric Protein as a Vaccine

Infectivity of the mice immunized with AlpA/CTXA2B chimeric protein and AlpA against H. pylori was investigated in an analogous manner as in Example 9-1, to determine the effect of AlpA/CTXA2B as a potential vaccine for H. pylori, with an exception that AlpA/CTXA2B chimeric protein was employed instead of UreB/CTXA2B. As a result, it was determined that the experimenatl groups administered with AlpA/CTXA2B and AlpA showed prevention rate of 75% and 50%, respectively. On the other hand, all mice of the control group administered with only a physiological saline were infected with H. pylori, which showed no preventive effect.

### EXAMPLE 9-4

### Effect of the AlpB/CTXA2B Chimeric Protein as a Vaccine

Infectivity of the mice immunized with AlpB/CTXA2B chimeric protein and AlpB against H. pylori was investigated in an analogous manner as in Example 9-1, to determine the effect of AlpB/CTXA2B as a potential vaccine for H. pylori, with an exception that AlpB/CTXA2B chimeric protein was employed instead of UreB/CTXA2B. As a result, it was determined that the experimenatl groups administered with AlpB/CTXA2B and AlpA showed prevention rate of 65% and 53%, respectively. On the other hand, all mice of the control group administered with only a physiological saline were infected with H. pylori, which showed no preventive effect.

### EXAMPLE 9-5

### Effect of the FliQ/CTXA2B Chimeric Protein as a Vaccine

Infectivity of the mice immunized with FliQ/CTXA2B chimeric protein and FliQ against H. pylori was investigated in an analogous manner as in Example 9-1, to determine the effect of FliQ/CTXA2B as a potential vaccine for H. pylori, with an exception that FliQ/CTXA2B chimeric protein was employed instead of FliQ/CTXA2B. As a result, it was determined that the experimenatl groups administered with FliQ/CTXA2B and FliQ showed prevention rate of 75% and 57%, respectively. On the other hand, all mice of the control group administered with only a physiological saline were infected with H. pylori, which showed no preventive effect.

### EXAMPLE 9-6

### Effect of the BabA1/CTXA2B Chimeric Protein as a Vaccine

Infectivity of the mice immunized with BabA1/CTXA2B chimeric protein and BabA1 against H. pylori was investigated in an analogous manner as in Example 9-1, to determine the effect of BabA1/CTXA2B as a potential vaccine for H. pylori, with an exception that BabA1/CTXA2B chimeric protein was employed instead of UreB/CTXA2B. As a result, it was determined that the experimenatl groups administered with BabA1/CTXA2B and BabA1 showed prevention rate of 80% and 57%, respectively. On the other hand, all mice of the control group administered with only a physiological saline were infected with H. pylori, which showed no preventive effect.

### EXAMPLE 9-7

### Effect of the BabA2/CTXA2B Chimeric Protein as a Vaccine

Infectivity of the mice immunized with BabA2/CTXA2B chimeric protein and BabA2 against H. pylori was investigated in an analogous manner as in Example 9-1, to determine the effect of BabA2/CTXA2B as a potential vaccine for H. pylori, with an exception that BabA2/CTXA2B chimeric protein was employed instead of UreB/CTXA2B. As a result, it was determined that the experimenatl groups administered with BabA2/CTXA2B and BabA2 showed prevention rate of 75% and 55%, respectively. On the other hand, all mice of the control group administered with only a physiological saline were infected with H. pylori, which showed no preventive effect.

### EXAMPLE 9-8

### Effect of the UreC/CTXA2B Chimeric Protein as a Vaccine

Infectivity of the mice immunized with UreC/CTXA2B chimeric protein and UreC against H. pylori was investigated in an analogous manner as in Example 9-1, to determine the effect of UreC/CTXA2B as a potential vaccine for H. pylori, with an exception that UreC/CTXA2B chimeric protein was employed instead of UreB/CTXA2B. As a result, it was determined that the experimenatl groups administered with UreC/CTXA2B and UreC showed prevention rate of 78% and 60%, respectively. On the other hand, all mice of the control group administered with only a physiological saline were infected with H. pylori, which showed no preventive effect.

### EXAMPLE 9-9

### Effect of the UreD/CTXA2B Chimeric Protein as a Vaccine

Infectivity of the mice immunized with UreD/CTXA2B chimeric protein and UreD against H. pylori was investigated in an analogous manner as in Example 9-1, to determine the effect of UreD/CTXA2B as a potential vaccine for H. pylori, with an exception that UreD/CTXA2B chimeric protein was employed instead of UreB/CTXA2B. As a result, it was determined that the experimenatl groups administered with UreD/CTXA2B and UreD showed prevention rate of 70% and 52%, respectively. On the other hand, all mice of the control group administered with only a physiological saline were infected with H. pylori, which showed no preventive effect.

### EXAMPLE 9-10

### Effect of the UreA/CTXA2B Chimeric Protein as a Vaccine

Infectivity of the mice immunized with UreA/CTXA2B chimeric protein and UreA against H. pylori was investigated in an analogous manner as in Example 9-1, to determine the effect of UreA/CTXA2B as a potential vaccine for H. pylori, with an exception that UreA/CTXA2B chimeric protein was employed instead of UreB/CTXA2B. As a result, it was determined that the experimenatl groups administered with UreA/CTXA2B and UreA showed prevention rate of 70% and 50%, respectively. On the other hand, all mice of the control group administered with only a physiological saline were infected with H. pylori, which showed no preventive effect.

### EXAMPLE 9-11

### Effect of the SodB/CTXA2B Chimeric Protein as a Vaccine

Infectivity of the mice immunized with SodB/CTXA2B chimeric protein and SodB against H. pylori was investigated in an analogous manner as in Example 9-1, to determine the effect of SodB/CTXA2B as a potential vaccine for H. pylori, with an exception that SodB/CTXA2B chimeric protein was employed instead of UreB/CTXA2B. As a result, it was determined that the experimenatl groups administered with SodB/CTXA2B and SodB showed prevention rate of 70% and 55%, respectively. On the other hand, all mice of the control group administered with only a physiological saline were infected with H. pylori, which showed no preventive effect.

### EXAMPLE 9-12

### Effect of the UreI/CTXA2B Chimeric Protein as a Vaccine

Infectivity of the mice immunized with UreI/CTXA2B chimeric protein and UreI against H. pylori was investigated in an analogous manner as in Example 9-1, to determine the effect of UreI/CTXA2B as a potential vaccine for H. pylori, with an exception that UreI/CTXA2B chimeric protein was employed instead of UreB/CTXA2B. As a result, it was determined that the experimenatl groups administered with UreI/CTXA2B and UreI showed prevention rate of 65% and 53%, respectively. On the other hand, all mice of the control group administered with only a physiological saline were infected with H. pylori, which showed no preventive effect.

### EXAMPLE 9-13

### Effect of the UreE/CTXA2B Chimeric Protein as a Vaccine

Infectivity of the mice immunized with UreE/CTXA2B chimeric protein and UreE against H. pylori was investigated in an analogous manner as in Example 9-1, to determine the effect of UreE/CTXA2B as a potential vaccine for H. pylori, with an exception that UreE/CTXA2B chimeric protein was employed instead of UreB/CTXA2B. As a result, it was determined that the experimenatl groups administered with UreE/CTXA2B and UreI showed prevention rate of 70% and 55%, respectively. On the other hand, all mice of the control group administered with only a physiological saline were infected with H. pylori, which showed no preventive effect.

### EXAMPLE 9-14

### Effect of the UreF/CTXA2B Chimeric Protein as a Vaccine

Infectivity of the mice immunized with UreF/CTXA2B chimeric protein and UreF against H. pylori was investigated in an analogous manner as in Example 9-1, to determine the effect of UreF/CTXA2B as a potential vaccine for H. pylori, with an exception that UreF/CTXA23 chimeric protein was employed instead of UreB/CTXA2B. As a result, it was determined that the experimenatl groups administered with UreF/CTXA2B and UreF showed prevention rate of 75% and 55%, respectively. On the other hand, all mice of the control group administered with only a physiological saline were infected with H. pylori, which showed no preventive effect.

### EXAMPLE 9-15

### Effect of the UreG/CTXA2B Chimeric Protein as a Vaccine

Infectivity of the mice immunized with UreG/CTXA2B chimeric protein and UreG against H. pylori was investigated in an analogous manner as in Example 9-1, to determine the effect of UreG/CTXA2B as a potential vaccine for H. pylori, with an exception that UreG/CTXA2B chimeric protein was employed instead of UreB/CTXA2B. As a result, it was determined that the experimenatl groups administered with UreG/CTXA2B and UreG showed prevention rate of 78% and 53%, respectively. On the other hand, all mice of the control group administered with only a physiological saline were infected with H. pylori, which showed no preventive effect.

### EXAMPLE 9-16

### Effect of the UreH/CTXA2B Chimeric Protein as a Vaccine

Infectivity of the mice immunized with UreH/CTXA2B chimeric protein and UreH against H. pylori was investigated in an analogous manner as in Example 9-1, to determine the effect of UreH/CTXA2B as a potential vaccine for H. pylori, with an exception that UreH/CTXA2B chimeric protein was employed instead of UreB/CTXA2B. As a result, it was determined that the experimenatl groups administered with UreH/CTXA2B and UreH showed prevention rate of 65% and 45%, respectively. On the other hand, all mice of the control group administered with only a physiological saline were infected with H. pylori, which showed no preventive effect.

### EXAMPLE 9-17

### Effect of the FlaA/CTXA2B Chimeric Protein as a Vaccine

Infectivity of the mice immunized with FlaA/CTXA2B chimeric protein and FlaA against H. pylori was investigated in an analogous manner as in Example 9-1, to determine the effect of FlaA/CTXA2B as a potential vaccine for H. pylori, with an exception that FlaA/CTXA2B chimeric protein was employed instead of UreB/CTXA2B. As a result, it was determined that the experimenatl groups administered with FlaA/CTXA2B and FlaA showed prevention rate of 70% and 52%, respectively. On the other hand, all mice of the control group administered with only a physiological saline were infected with H. pylori, which showed no preventive effect.

### EXAMPLE 9-18

### Effect of the FlaB/CTXA2B Chimeric Protein as a Vaccine

Infectivity of the mice immunized with FlaB/CTXA2B chimeric protein and FlaB against H. pylori was investigated in an analogous manner as in Example 9-1, to determine the effect of FlaB/CTXA2E3 as a potential vaccine for H. pylori, with an exception that FlaB/CTXA2B chimeric protein was employed instead of UreB/CTXA2B. As a result, it was determined that the experimenatl groups administered with FlaB/CTXA2B and FlaB showed prevention rate of 78% and 50%, respectively. On the other hand, all mice of the control group administered with only a physiological saline were infected with H. pylori, which showed no preventive effect.

### EXAMPLE 9-19

### Effect of the CatA/CTXA2B Chimeric Protein as a Vaccine

Infectivity of the mice immunized with CatA/CTXA2B chimeric protein and CatA against H. pylori was investigated in an analogous manner as in Example 9-1, to determine the effect of CatA/CTXA2B as a potential vaccine for H. pylori, with an exception that CatA/CTXA2B chimeric protein was employed instead of UreB/CTXA2B. As a result, it was determined that the experimenatl groups administered with CatA/CTXA2B and CatA showed prevention rate of 75% and 50%, respectively. On the other hand, all mice of the control group administered with only a physiological saline were infected with H. pylori, which showed no preventive effect.

### EXAMPLE 9-20

### Effect of the VacA/CTXA2B Chimeric Protein as a Vaccine

Infectivity of the mice immunized with VacA/CTXA2B chimeric protein and VacA against H. pylori was investigated in an analogous manner as in Example 9-1, to determine the effect of VacA/CTXA2B as a potential vaccine for H. pylori, with an exception that VacA/CTXA2B chimeric protein was employed instead of UreB/CTXA2B. As a result, it was determined that the experimenatl groups administered with VacA/CTXA2B and VacA showed prevention rate of 68% and 53%, respectively. On the other hand, all mice of the control group administered with only a physiological saline were infected with H. pylori, which showed no preventive effect.

### EXAMPLE 9-21

### Effect of the BabB/CTXA2B Chimeric Protein as a Vaccine

Infectivity of the mice immunized with BabB/CTXA2B chimeric protein and BabB against H. pylori was investigated in an analogous manner as in Example 9-1, to determine the effect of BabB/CTXA2B as a potential vaccine for H. pylori, with an exception that BabB/CTXA2B chimeric protein was employed instead of UreB/CTXA2B. As a result, it was determined that the experimenatl groups administered with BabB/CTXA2B and BabB showed prevention rate of 72% and 53%, respectively. On the other hand, all mice of the control group administered with only a physiological saline were infected with H. pylori, which showed no preventive effect.

As shown in Examples 9-1 to 9-21, it was clearly demonstrated that: the chimeric proteins of antigenic proteins of H. pylori and A2 and B subunits of Vibrio cholerae, can induce specific antibodies neutralizing H. pylori and be used as a preventive or therapeutic vaccine for H. pylori-associated diseases.

### PREPARATIVE EXAMPLE 1

### Solution Containing UreB/CTXA2B Chimeric Protein

| | |
|---|---|
| UreB/CTXA2B chimeric protein | 100 μg |
| 0.6M sodium bicarbonate | 250 μl |
| Distilled water | 250 μl |
| Total | 500 μl |

A solution containing UreB/CTXA2B chimeric protein was prepared as described above.

### PREPARATIVE EXAMPLE 2

### Solution Containing CagA/CTXA2B Chimeric Protein

| | |
|---|---|
| CagA/CTXA2E chimeric protein | 100 μg |
| 0.6M sodium bicarbonate | 250 μl |
| Distilled water | 250 μl |
| Total | 500 μl |

A solution containing CagA/CTXA2B chimeric protein was prepared as described above.

### PREPARATIVE EXAMPLE 3

### Solution Containing AlpA/CTXA2B Chimeric Protein

| | |
|---|---|
| AlpA/CTXA2B chimeric protein | 100 μg |
| 0.6M sodium bicarbonate | 250 μl |
| Distilled water | 250 μl |
| Total | 500 μl |

A solution containing AlpA/CTXA2B chimeric protein was prepared as described above.

### PREPARATIVE EXAMPLE 4

### Solution Containing AlpB/CTXA2B Chimeric Protein

| | |
|---|---|
| AlpB/CTXA2B chimeric protein | 100 μg |
| 0.6M sodium bicarbonate | 250 μl |
| Distilled water | 250 μl |
| Total | 500 μl |

A solution containing AlpB/CTXA2B chimeric protein was prepared as described above.

### PREPARATIVE EXAMPLE 5

### Solution Containing FliQ/CTXA2B Chimeric Protein

| | |
|---|---|
| FliQ/CTXA2B chimeric protein | 100 μg |
| 0.6M sodium bicarbonate | 250 μ1 |
| Distilled water | 250 μl |
| Total | 500 μl |

A solution containing FliQ/CTXA2B chimeric protein was prepared as described above.

### PREPARATIVE EXAMPLE 6

### Solution Containing BabA1/CTXA2B Chimeric Protein

| | |
|---|---|
| BabAl/CTXA2B chimeric protein | 100 μg |
| 0.6M sodium bicarbonate | 250 μl |
| Distilled water | 250 μl |
| Total | 500 μl |

A solution containing BabA1/CTXA2B chimeric protein was prepared as described above.

### PREPARATIVE EXAMPLE 7

### Solution Containing BabA2/CTXA2B Chimeric Protein

| | |
|---|---|
| BabA2/CTXA2B chimeric protein | 100 μg |
| 0.6M sodium bicarbonate | 250 μl |
| Distilled water | 250 μl |
| Total | 500 μl |

A solution containing BabA2/CTXA2B chimeric protein was prepared as described above.

### PREPARATIVE EXAMPLE 8

### Solution Containing UreC/CTXA2B Chimeric Protein

| | |
|---|---|
| UreC/CTXA2B chimeric protein | 100 μg |
| 0.6M sodium bicarbonate | 250 μl |
| Distilled water | 250 μl |
| Total | 500 μl |

A solution containing UreC/CTXA2B chimeric protein was prepared as described above.

### PREPARATIVE EXAMPLE 9

### Solution Containing UreD/CTXA2B Chimeric Protein

| | |
|---|---|
| UreD/CTXA2B chimeric protein | 100 μg |
| 0.06M sodium bicarbonate | 250 μl |
| Distilled water | 250 μl |
| Total | 500 μl |

A solution containing UreD/CTXA2B chimeric protein was prepared as described above.

### PREPARATIVE EXAMPLE 10

### Solution Containing UreA/CTXA2B Chimeric Protein

| | |
|---|---|
| UreA/CTXA2B chimeric protein | 100 μg |
| 0.6M sodium bicarbonate | 250 μl |
| Distilled water | 250 μl |
| Total | 500 μl |

A solution containing UreA/CTXA2B chimeric protein was prepared as described above.

### PREPARATIVE EXAMPLE 11

### Solution Containing SodB/CTXA2B Chimeric Protein

| | |
|---|---|
| SodB/CTXA2B chimeric protein | 100 μg |
| 0.6M sodium bicarbonate | 250 μl |
| Distilled water | 250 μl |
| Total | 500 μl |

A solution containing SodB/CTXA2B chimeric protein was prepared as described above.

### PREPARATIVE EXAMPLE 12

### Solution containing UreI/CTXA2B Chimeric Protein

| | |
|---|---|
| UreI/CTXA2B chimeric protein | 100 μg |
| 0.6M sodium bicarbonate | 250 μl |
| Distilled water | 250 μl |
| Total | 500 μl |

A solution containing UreI/CTXA2B chimeric protein was prepared as described above.

### PREPARATIVE EXAMPLE 13

### Solution Containing UreE/CTXA2B Chimeric Protein

| | |
|---|---|
| UreE/CTXA2B chimeric protein | 100 μg |
| 0.6M sodium bicarbonate | 250 μl |
| Distilled water | 250 μl |
| Total | 500 μl |

A solution containing UreE/CTXA2B chimeric protein was prepared as described above.

### PREPARATIVE EXAMPLE 14

### Solution Containing UreF/CTXA2B Chimeric Protein

| | |
|---|---|
| UreF/CTXA2B chimeric protein | 100 μg |
| 0.6M sodium bicarbonate | 250 μl |
| Distilled water | 250 μl |
| Total | 500 μl |

A solution containing UreF/CTXA2B chimeric protein was prepared as described above.

### PREPARATIVE EXAMPLE 15

### Solution Containing UreG/CTXA22 Chimeric Protein

| | |
|---|---|
| UreG/CTXA2B chimeric protein | 100 μg |
| 0.6M sodium bicarbonate | 250 μl |
| Distilled water | 250 μl |
| Total | 500 μl |

A solution containing UreG/CTXA2B chimeric protein was prepared as described above.

### PREPARATIVE EXAMPLE 16

### Solution Containing UreH/CTXA2B Chimeric Protein

| | |
|---|---|
| UreH/CTXA2B chirneric protein | 100 μg |
| 0.6 M sodium bicarbonate | 250 μl |
| Distilled water | 250 μl |
| Total | 500 μl |

A solution containing UreH/CTXA2B chimeric protein was prepared as described above.

### PREPARATIVE EXAMPLE 17

### Solution Containing FlaA/CTXA2B Chimeric Protein

| | |
|---|---|
| PlaA/CTXA2B chimeric protein | 100 μg |
| 0.6M sodium bicarbonate | 250 μl |
| Distilled water | 250 μl |
| Total | 500 μl |

A solution containing FlaA/CTXA2B chimeric protein was prepared as described above.

### PREPARATIVE EXAMPLE 18

### Solution Containing FlaB/CTXA2B Chimeric Protein

| | |
|---|---|
| FlaB/CTXA2B chimeric protein | 100 μg |
| 0.6 M sodium bicarbonate | 250 μl |
| Distilled water | 250 μl |
| Total | 500 μl |

A solution containing FlaB/CTXA2B chimeric protein was prepared as described above.

### PREPARATIVE EXAMPLE 19

### Solution Containing CatA/CTXA2B Chimeric Protein

| | |
|---|---|
| CatA/CTXA2B chimeric protein | 100 μg |
| 0.6 M sodium bicarbonate | 250 μl |
| Distilled water | 250 μl |
| Total | 500 μl |

A solution containing CatA/CTXA2B chimeric protein was prepared as described above.

### PREPARATIVE EXAMPLE 20

### Solution Containing VacA/CTXA2B Chimeric Protein

| | |
|---|---|
| VacA/CTXA2B chimeric protein | 100 μg |
| 0.6 M sodium bicarbonate | 250 μl |
| Distilled water | 250 μl |
| Total | 500 μl |

A solution containing VacA/CTXA2B chimeric protein was prepared as described above.

### PREPARATIVE EXAMPLE 21

### Solution Containing BabB/CTXA2B Chimeric Protein

| | |
|---|---|
| BabB/CTXA2B chimeric protein | 100 μg |
| 0.6 M sodium bicarbonate | 250 μl |
| Distilled water | 250 μl |
| Total | 500 μl |

A solution containing BabB/CTXA2B chimeric protein was prepared as described above.

As clearly illustrated and demonstrated as aboves, the present invention provides a series of recombinant DNAs which are prepared by ligating antigenic determinant coding genes of H. pylori and A2 and B subunit genes of Vibrio cholerae toxin, and a process for preparing the chimeric proteins of antigenic proteins of H. pylori and A2 and B subunits of Vibrio cholerae toxin, employing recombinant microorganisms transformed with the recombinant expression vectors comprising the recombinant DNAs. The recombinant DNAs which are designed for convenient expression and gene manipulation, can express chimeric proteins having excellent immunogenicity to H. pylori, which are stable in stomach, and penetrate mucous membrane of intestines easily, finally to stimulate production of sIgA. Accordingly, the chimeric proteins expressed from the recombinant DNAs may be used as an active ingredient of the diagnostic kit for H. pylori infection and preventive or therapeutic vaccine for H. pylori-associated diseases, and may be used in the production of anti-H. pylori antibody.

What is claimed is:

1. A recombinant DNA comprising a fusion gene which is prepared by ligating antigenic determinant coding gene of Helicobacter pylori and A2 and B subunit genes of Vibrio cholerae toxin.

2. The recombinant DNA of claim 1, wherein the antigenic determinant coding gene of Helicobacter pylori is selected from the group consisting of ureB, cagA, alpA, alpB, fliQ, babA1, babA2, ureC, ureD, ureA, sodB, ureI, ureE, ureF, ureG, ureH, flaA, flaB, catA, vacA and babB.

3. The recombinant DNA of claim 1, wherein the fusion gene which is prepared by ligating ureB gene of Helicobacter pylori and A2 and B subunit genes of Vibro cholerae toxin, has a nucleotide sequence represented as following, or its functional equivalents:
atgaaaaaga ttagcagaaa agaatatgct tctatgtatg gccctactac aggcgataaa gtgagattgg gcgatacaga cttgatcgct gaagtagaac atgactacac catttatggt gaagagctta aattcggcgg cggtaaaacc ctaagagaag gcatgagcca atctaacaac cctagcaaag aagaactgga tctaatcatc actaacgctt taatcgtgga ttacaccggt atttataaag cggatattgg tattaaagat ggcaaaatcg ctggcattgg taaaggcggt aacaaagaca cgcaagatgg cgttaaaaac aatcttagcg tgggtcctgc tactgaagcc ttagccggtg aaggtttgat tgtaactgct ggtggtattg acacacacat ccacttcatc tccccccaac aaatccctac agcttttgca agcggtgtaa caaccatgat tggtggcgga actggccctg ctgatggcac taacgcaacc actatcactc caggtagaag aaatttaaaa ttcatgctca gagcggctga agaatattct atgaactttg gtttcttggc taaaggtaac gcttctaacg atgcaagctt agccgatcaa attgaagctg gtgcgattgg ccttaaaatc cacgaagact ggggcaccac tccttctgca atcaatcatg cgttagatgt tgcggacaaa tacgatgtgc aagtcgctat ccacacagac actttgaatg aagccggttg cgtggaagac actatggcag ctattgccgg acgcactatg cacacttacc acactgaagg cgctggcggc ggacacgctc ctgatattat taaagtggcc ggtgaacaca acatcctacc cgcttccact aaccccacta tccctttcac cgtgaataca gaagccgaac acatggacat gcttatggtg tgccaccact tggataaaag cattaaagaa gatgtccagt tcgctgattc aaggattcgc cctcaaacca ttgcggctga agacactttg catgacatgg ggattttctc aatcactagt tctgactctc aagcgatggg ccgtgtgggt gaagttatca ctagaacttg gcaaacagct gacaaaaata aaaaagaatt tggccgcttg aaagaagaaa aaggcgataa cgacaacttc aggatcaaac gctacttgtc taaatacacc attaacccag cgatcgctca tgggattagc gagtatgtcg gttctgtaga agtgggcaaa gtggctgact tggtattgtg gagtcccgca ttctttggtg tgaaacccaa catgatcatc aaaggcgggt tcatcgcatt gagtcaaatg ggtgatgcga acgcttctat ccctacccca caaccagttt attacagaga aatgttcgct catcatggta aagctaaata cgatgcaaac atcacttttg tgtctcaagc ggcttatgac aaaggcatta aagaagaatt agggcttgaa agacaagtgt tgccggtaaa aaattgcaga aatatcacta aaaaagacat gcaattcaac gacactaccg ctcacattga agtcaattct gaaacttacc atgtgttcgt ggatggcaaa gaagtaactc taaaccagcc aataaagtga gaattcgaag agccgtggat tcatcatgca ccgccgggtt gtgggaatgc tccaagatca tcgatcagta atacttgcga tgaaaaaacc caaagtctag gtgtaaaatt ccttgacgaa taccaatcta aagttaaaag acaaatattt tcaggctatc aatctgatat tgatacacat aatagaatta aggatgaatt aatgattaaa ttaaaatttg gtgttttttt tacagtttta ctatcttcag catatgcaca tggaacacct caaaatatta ctgatttgtg tgcagaatca cacaacacac aaatatatac gctaaatgat aagatatttt cgtatacaga atctctagct ggaaaaagag agatggctat cattactttt aagaatggtg caatttttca agtagaagta ccaagtagtc aacatataga ttcacaaaaa aaagcgattg aaaggatgaa ggataccctg aggattgcat atcttactga agctaaagtc gaaaagttat gtgtatggaa taataaaacg cctcatgcga ttgccgcaat tagtatggca aattaagata taaaaagccc acctcagtgg gcttttttgt ggttcgatga tgagaagcaa ccgttttgcc caaacatgta ttactgcaag tatgatgttt ttattccaca tccttagtgc gtattatgtg ctgca.

4. The recombinant DNA of claim 1, wherein the fusion gene which is prepared by ligating cagA gene of Helicobacter pylori and A2 and B subunit genes of Vibro cholerae toxin, has a nucleotide sequence represented as following, or its functional equivalents:
atgactaacg aaaccattga ccaacaacca caaaccgaag cggcttttaa cccgcagcaa tttatcaata atcttcaagt agcttttctt aaagttgata acgctgtcgc ttcatacgat cctgatcaaa aaccaatcgt tgataagaac gatagggata acaggcaagc ttttgaagga atctcgcaat taagggaaga atactccaat aaagcgatca aaaatcctac caaaaagaat cagtattttt cagactttat caataagagc aatgatttaa tcaacaaaga caatctcatt gatgtagaat cttccacaaa gagctttcag aaatttgggg atcagcgtta ccgaattttc acaagttggg tgtcccatca aaacgatccg tctaaaatca acacccgatc gatccgaaat tttatggaaa atatcataca accccctatc cttgatgata aagagaaagc ggagtttttg aaatctgcca aacaatcttt tgcaggaatc attataggga atcaaatccg aacggatcaa aagttcatgg gcgtgtttga tgagtccttg aaagaaaggc aagaagcaga aaaaaatgga gagcctactg gtggggattg gttggatatt tttctctcat ttatatttga caaaaaacaa tcttctgatg tcaaagaagc aatcaatcaa gaaccagttc cccatgtcca accagatata gccactacca ccaccgacat acaaggctta ccgcctgaag ctagagattt acttgatgaa aggggtaatt tttctaaatt cactcttggc gatatggaaa tgttagatgt tgagggagtc gctgacattg atcccaatta caagttcaat caattattga ttcacaataa cgctctgtct tctgtgttaa tggggagtca taatggcata gaacctgaaa aagtttcatt gttgtatggg ggcaatggtg gtcctggagc taggcatgat tggaacgcca ccgttggtta taaagaccaa caaggcaaca atgtggctac aataattaat gtgcatatga aaaacggcag tggcttagtc atagcaggtg gtgagaaagg gattaacaac cctagttttt atctctacaa agaagaccaa ctcacaggct cacaacgagc attaagtcaa gaagagatcc aaaacaaaat agatttcatg gaatttcttg cacaaaataa tgctaaatta gacaacttga gcgagaaaga gaaggaaaaa ttccgaactg agattaaaga tttccaaaaa gactctaagg cttatttaga cgccctaggg aatgatcgta ttgcttttgt ttctaaaaaa gacacaaaac attcagcttt aattactgag tttggtaatg gggatttgag ctacactctc aaagattatg ggaaaaaagc agataaagct ttagataggg agaaaaatgt tactcttcaa ggtagcctaa aacatgatgg cgtgatgttt gttgattatt ctaatttcan atacaccaac gcctccaaga atcccaataa gggtglaggc gttacgaatg gcgtttccca tttagaagta ggctttaaca aggtagctat ctttaatttg cctgatttaa ataatctcgc tatcactagt ttcgtaaggc ggaatttaga ggataaacta accactaaag gattgtcccc acaagaagct aataagctta tcaaagattt ttlgagcagc aacaaagaat tggttggaaa aactttaaac ttcaataaag ctgtagctga cgctaaaaac acaggcaatt atgatgaagt gaaaaaagct cagaaagatc ttgaaaaatc tctaaggaaa cgagagcatt tagagaaaga agtagagaaa aaattggaga gcaaaagcgg caacaaaaat aaaatggaag caaaagstca agctaacagc caaaaagatg agatttttgc gttgatcaat aaagaggcta atagagacgc aagagcaatc gcttacgctc agaatcttaa aggcatcaaa agggaattgt ctgataaact tgaaaatgtc aacaagaatt tgaaagactt tgataaatct tttgatgaat tcaaaaatgg caaaaataag gaittcagca aggcagaaga aacactaaaa gcccttaaag gttcggtgaa agatttaggt atcaatccag aatggatttc aaaagttgaa aaccttaatg cagctttgaa tgaattcaaa aatggcaaaa ataaggattt cagcaaggta acgcaagcaa aaagcgacct tgaaaattcc gttaaagatg igatcatcaa tcaaaaggta acggataaag ttgataatct caatcaagcg gtatcagtgg ctaaagcaac gggtgatttc agtagggtag agcaagcgtt agccgatctc aaaaattict caaaggagca attggcccaa caagctcaaa aaaatgaaag tctcaatgct agaaaaaaat ctgaaatata tcaatccgtt aagaatggtg tgaatggaac cctagtcggt aatgggttat ctcaagcaga agccacaact ctttctaaaa acttttcgga catcaagaaa gagttgaatg caaaacttgg aaatttcaat aacaataaca ataatggact caaaaacgaa cccatttatg ctaaagttaa taaaaagaaa gcagggcaag cagctagcct tgaagaaccc atttacgctc aagttgctaa aaaggtaaat gcaaaaattg accgactcaa tcaaatagca agtggtttgg gtgttgtagg gcaagcagcg ggcttccctt tgaaaaggca tgataaagtt gatgatctca gtaaggtagg gctttcaagg aatcaagaat tggctcagaa aattgacaat ctcaatcaag cggtatcaga agctaaagca ggtttttttg gcaatctaga gcaaacgata gacaagctca aagattctac aaaacacaat cccatgaatc tatgggttga aagtgcaaaa aaagtacctg ctagtttgtc agcgaaacta gacaattacg ctactaacag ccacatacgc attaatagca atatcaaaaa tggagcaatc aatgaaaaag cgaccggcat gctaacgcaa aaaaaccctg agtggctcaa gctcgtgaat gataagatag ttgcgcataa tgtaggaagc gttcctttgt cagagtatga taaaattggc ttcaaccaga agaatatgaa agattattct gattcgttca agttttccac caagttgaac aatgctgtaa aagacactaa ttctggcttt acgcaatttt taaccaatgc attttctaca gcatcttatt actgcttggc gagagaaaat gcggagcatg gaatcaagaa cgttaataca aaaggtggtt tccaaaaatc ttaagaattc gaagagccgt ggattcatca tgcaccgccg ggttgtggga atgctccaag atcatcgatc agtaatactt gcgatgaaaa aacccaaagt ctaggtgtaa aattccttga cgaataccaa tctaaagtta aaagacaaat attttcaggc tatcaatctg atattgatac acataataga attaaggatg aattaatgat taaattaaaa tttggtgttt tttttacagt tttactatct tcagcatatg cacatggaac acctcaaaat attactgatt tgtgtgcaga atcacacaac acacaaatat atacgctaaa tgataagata ttttcgtata cagaatctct agctggaaaa agagagatgg ctatcattac ttttaagaat ggtgcaattt ttcaagtaga agtaccaagt agtcaacata tagattcaca aaaaaaagcg attgaaagga tgaaggatac cctgaggatt gcatatctta ctgaagctaa agtcgaaaag ttatgtgtat ggaataataa aacgcctcat gcgattgccg caattagtat ggcaaattaa gatataaaaa gcccacctca gtgggctttt ttgtggttcg atgatgagaa gcaaccgttt tgcccaaaca tgtattactg caagtatgat gtttttattc cacatcctta gtgcgtatta tgtgctgca.

5. A chimeric protein consisting of antigenic protein of Helicobacter pylori and A2 and B subunits of Vibrio cholerae toxin which has an amino acid sequence deducable from the recombinant DNA of claim 1, or its functional equivalents.

6. The chimeric protein of claim 5, wherein the antigenic protein of Helicobacter pylori is selected from the group consisting of UreB, CagA, AlpA, AlpB, FliQ, BabA1, BabA2, UreC, UreD, UreA, SodB, UreI, UreE, UreF, UreG, UreH, FlaA, FlaB, CatA, VacA and BabB.

7. A chimeric protein which is prepared by ligating UreB of Helicobacter pylori and A2 and B subunits of Vibrio cholerae toxin, has an amino acid sequence represented as following, or its functional equivalents:
MKISRKEYA SMYGPTTGDK VRLGDTDLIA EVEHDYTIYG EELKFGGGKT LREGMSQSNN PSKEELDLII TNALIVDYTG IYKADlGIKD GKIAGIGKGG NKDTQDGVKN NLSGPATEA LAGEGLIVTA GGIDTHIHFI SPQQIPTAFA SGVTTNIIGGG TGPADGTNAT TITPGRRNLK FMLRAAEEYS MNFGFLAKGN ASNDASLADQ IEAGAIGLKI HEDWGTTPSA INHALDVADK YDVQVAIHTD TLNEAGCVED TMAAIAGRTM HTYHTEGAGG GHAPDIIKVA GEHNILPAST NPTIPFTVNT EAEHMDMLMV CHHLDKSIKE DVQFADSRIR PQTIAAEDTL HDMGIFSITS SDSQAMGRVG EVITRTWQTA DKNKKEFGRL KEEKGDNDNF RIKRYLSKYT INPAIAHGIS EYVGSVEVGK VADLVLWSPA FFGVKPNMII KGGFIALSQM GDANASIPTP QPVYYREMFA HHGKAKYDAN ITFVSQAAYD KGIKEELGLE RQVLPVKNCR NITKKDMQFN DTTAHIEVNS ETYHVFVDGK EVTLNQPIKE FEEPWIHHAP PGCGNAPRSS ISNTCDEKTQ SLGVKFLDEY QSKVKRQIFS GYQSDIDTHN RIKDELMIKL KFGVFFTVLL SSAYAHGTPQ NITDLCAESH NTQIYTLNDK IFSYTESLAG KREMAIITFK NGAIFQVEVP SSQHIDSQKK AIERMKDTLR IAYLTEAKVE KLCVWNNKTP HAIAAISMAN.

8. A chimeric protein which is prepared by ligating CagA of Helicobacter pylori and A2 and B subunits of Vibrio cholerae toxin, has an amino acid sequence represented as following, or its functional equivalents:
MTNETIDQQP QTEAAFNPQQ FINNLOVAFL KVDNAVASYD PDQKPIVDKN DRDNRQAFEG ISQLREEYSN KAIKNPTKKN QYFSDFINKS NDLINKDNLI DVESSTKSFQ KFGDQRYRIF TSWVSHQNDP SKINTRSIRN FMENIIQPPI LDDKEKAEFL KSAKQSFAGI IIGNQIRTDQ KFMGVFDESL KERQEAEKNG EPTGGDWLDI FLSFIFDKKQ SSDVKEAINQ EPVPHVQPDI ATTTTDIQGL PPEARDLLDE RGNFSKFTLG DMEMLDVEGV ADIDPNYKFN QLLIHNNALS SVLMGSHNGI EPEKVSLLYG GNGGPGARHD WNATVGYKDQ QGNNVATIIN VHMKNGSGLV IAGGEKGINN PSFYLYKEDQ LTGSQRALSQ EEIQNKIDFM EFLAQNNAKL DNLSEKEKEK FRTEIKDFQK DSKAYLDALG NDRIAFVSKK DTKHSALITE FGNGDLSYTL KDYGKKADKA LDREKNVTLQ GSLKHDGVMF VDYSNFKYTN ASKNPNKGVG VTNGVSHLEV GFNKVAIFNL PDLNNLAITS FVRRNLEDKL TTKGLSPQEA NKLIKDFLSS NKELVGKTLN FNKAVADAKN TGNYDEVKKA QKDLEKSLRK REHLEKEVEK KLESKSGNKN KMEAKAQANS QKDEIFALIN KEANRDARAI AYAQNLKGIK RELSDKLENV NKNLKDFDKS FDEFKNGKNK DFSKAEETLK ALKGSVKDLG INPEWISKVE NLNAALNEFK NGKNKDFSKV TQAKSDLENS VKDVIINQKV TDKVDNLNQA VSVAKATGDF SRVEQALADL KNFSKEQLAQ QAQKNESLNA RKKSEIYQSV KNGVNGTLVG NGLSQAEATT LSKNFSDIKK ELNAKLGNFN NNNNNGLKNE PIYAKVNKKK AGQAASLEEP IYAQVAKKVN AKIDRLNQIA SGLGVVGQAA GFPLKRHDKV DDLSKVGLSR NQELAQKIDN LNQAVSEAKA GFFGNLEQTI DKLKDSTKHN PMNLWVESAK KVPASLSAKL DNYATNSHIR INSNIKNGAI NEKATGMLTQ KNPEWLKLVN DKIVAHNVGS VPLSEYDKIG FNQKNMKDYS DSFKFSTKLN NAVKDTNSGF TQFLTNAFST ASYYCLAREN AEHGIKNVNT KGGFQKSEFE EFWIHHAPPG CGNAPRSSIS NTCDEKTQSL GVKFLDEYQS KVKRQIFSGY QSDIDTHNRI KDELMIKLKF GVFFTVLLSS AYAHGTPQNI TDLCAESHNT QIYTLNDKIF SYTESLAGKR EMAIITFKNG AIFQVEVPSS QHIDSQKKAI ERMKDTLRIA YLTEAKVEKL CVWNNKTPHA IAAISMAN.

9. A recombinant expression vector which comprises the recombinant DNA of claim 1, to express a chimeric protein consisting of antigenic protein of Helicobacter pylori and A2 and B subunits of Vibro cholerae toxin.

10. A recombinant expression vector which is capable of expressing a chimeric protein consisting of UreB of Helicobacter pylori and A2 and B subunits of Vibrio cholerae toxin, represented as following genetic map:

[chemical structure]

11. A recombinant expression vector which is capable of expressing a chimeric protein consisting of CagA of Helicobacter pylori and A2 and 1B subunits of Vibrio cholerae toxin, represented as following genetic map:

[chemical structure]

12. A recombinant Escherichia coli transformed with the recombinant expression vector of claim 9.

13. Escherichia coli DW/HU-044(KCCM-10124) which is capable of expressing a chimeric protein consisting of UreB of Helicobacter pylori and A2 and B subunits of Vibrio cholerae toxin.

14. Escherichia coli DW/HC-033(KCCM-10123) which is capable of expressing a chimeric protein consisting of CagA of Helicobacter pylori and A2 and B subunits of Vibrio cholerae toxin.

15. A process for preparing a chimeric protein which comprises the steps of:
culturing a microorganism transformed with the recombinant expression vector of claim 9; and,
recovering a chimeric protein consisting of antigenic protein of Helicobacter pylori and A2 and B subunits of Vibrio cholerae toxin.

16. The process for preparing a chimeric protein of claim 15, wherein the antigenic protein of Helicobacter pylori is selected from the group consisting of UreB, CagA, AlpA, AlpB, FliQ, BabA1, BabA2, UreC, UreD, UreA, SodB, UreI, UreE, UreF, UreG, UreH, FlaA, FlaB, CatA, VacA and BabB.

17. The process for preparing a chimeric protein of claim 15, wherein the microorganism is Escherichia coli DW/HU-044(KCCM-10124).

18. The process for preparing a chimeric protein of claim 15, wherein the microorganism is Escherichia coli DW/HC-033(KCCM-10123).

19. A chimeric protein which is prepared by the process comprising the steps of:
culturing a microorganism transformed with the recombinant expression vector of claim recombinant expression vector of claim 9; and,
recovering a chimeric protein consisting of antigenic protein of Helicobacter pylori and A2 and B subunits of Vibrio cholerae toxin.

20. The chimeric protein of claim 19, wherein the antigenic protein of Helicobacter pylori is selected from the group consisting of UreB, CagA, AlpA, AlpB, FliQ, BabA1, BabA2, UreC, UreD, UreA, SodB, UreI, UreE, UreF, UreG, UreH, FlaA, FlaB, CatA, VacA and BabB.

21. A preventive and therapeutic vaccine for Helicobacter pylori-associated diseases which comprises an active ingredient of a chimeric protein consisting of antigenic protein of Helicobacter pylori and A2 and B subunits of Vibro cholerae toxin, and its pharmaceutically acceptable carrier.

22. The preventive and therapeutic vaccine for Helicobacter pylori-associated diseases of claim 21, wherein the antigenic protein of Helicobacter pylori is selected from the group consisting of UreB, CagA, AlpA, AlpB, FliQ, BabA1, BabA2, UreC, UreD, UreA, SodB, UreI, UreE, UreF, UreG, UreH, FlaA, FlaB, CatA, VacA and BabB.

23. The preventive and therapeutic vaccine for Helicobacter pylori-associated diseases of claim 21, wherein the Helicobacter pylori-associated diseases comprise gastris, gastric ulcer, duodenal ulcer and gastric cancer.
